(19) United States
(12) Patent Application Publication
(10) Pub. No.: US 2023/0377699 A1
(43) Pub. Date: Nov. 23, 2023

(54) TECHNIQUES TO DETERMINE PATTERNS IN BLOOD GLUCOSE MEASUREMENT DATA AND A USER INTERFACE FOR PRESENTATION THEREOF

(71) Applicant: INSULET CORPORATION, Acton, MA (US)

(72) Inventors: Yuanzhi Zheng, Monrovia, CA (US); Gregory Stern, Lexington, MA (US); Bishal Bastola, Malden, MA (US); Kaycee Jarman, Winter Park, FL (US); Ethan Gromet, Laguna Beach, CA (US); Alicia Siman, Brooklyn, NY (US)

(21) Appl. No.: 17/746,461

(22) Filed: May 17, 2022

(51) Int. Cl. G16H 10/60; A61M 5/172

(52) U.S. Cl. CPC G16H 10/60; A61M 5/1723; A61M 2205/505; A61M 2230/201

(57) ABSTRACT

Disclosed are techniques, devices and computer products in which a processor is operable to access blood glucose measurement values and insulin data from a data warehouse memory. Based on the blood glucose measurement values the processor may be operable to identify high events that exceed an upper target blood glucose set point and low events in which blood glucose measurement values are below a lower target blood glucose set point. Rules may be applied to the high events and low events. Based on a result of the rules, high event patterns may be identified as well as a low event pattern. A respective pattern weight may be applied to each identified high event and each identified low and a graphical user interface may be populated with a number of high event patterns and for a number of low event patterns.

### BACKGROUND

Different systems are available for visualizing diabetes treatment program results and actions. An example is the Ambulatory Glucose Profile (AGP), which is a data visualization system that provides a user, such as a person with diabetes, with a dashboard that shows the user's blood glucose measurement data over a progression of days. However, the user is left to draw their own inferences from the data presented in the dashboard and/or rely on a healthcare provider to interpret the data.

In data visualization systems, such as AGP, daily glucose profiles are combined to provide a one day (24-hour) picture for presentation in the dashboard. The daily presentation shows the user's blood glucose measurement values as lines and, ideally, the lines stay within an outlined area (representing) that indicates the user's blood glucose measurements are staying within range (e.g., 10-25%) of the user's target setpoint. For example, the dashboard may present a middle line, a middle opacity, and a light opacity. The middle line is the median line where half of the number of blood glucose measurement values is above the middle line and the other half of the number of blood glucose measurement values is below the middle line. The middle opacity shows 50% of the blood glucose measurement values; ideally, the space between them is narrow indicating that the blood glucose measurement values are stable. The light opacity is an indication of 5% of blood glucose measurement values that are above (95% top line) and 5% of blood glucose measurement values that are below (5% bottom line). Any exceptions to the above are highlighted in red. The blood glucose data may also be aggregated over a period of time with daily data presented at a higher level as well as a presentation of the median of the aggregated data over the period of time.

While an AGP report provides a summary of information related to blood glucose information, it does not provide an analysis of the information or statistics to identify trends or patterns related to how well a user is staying within range of their target blood glucose set point and does not offer the analytics or visualization that enables improved presentation of the quantity of data available.

It would be beneficial to users to have a system that utilized techniques for data visualization of information drawn from a user's blood glucose measurement values as well as analytical techniques that are also incorporated into the data visualization.

### BRIEF SUMMARY

Disclosed is an example of a computer readable medium may be embodied with programming code that causes a computer to access blood glucose measurement values and insulin data from a data warehouse memory. Blood glucose measurement values that exceed an upper target blood glucose set point as a high events and blood glucose measurement values that are below a lower target blood glucose set point as low events may be identified. A high event overlap rule set may be applied to the high events and a low event overlap rule set may be applied to the low events. Based on the application of the high event overlap rule set to the high events and the low event overlap rule to the low events, overlapping high events and overlapping low events may be identified for analysis. Those high events that fall within a set high event overlap duration and day range may be identified as a high event pattern of the overlapping high events. The processor may be operable to identify as a low event pattern of the overlapping low events those low events that fall within a set low event overlap duration and day range. A respective pattern weight may be applied to each identified high event and each identified low event based on criteria specific to high events and specific to low events; and a graphical user interface may be populated with a number of high event patterns and for another number of low event patterns.

Disclosed is an example of another non-transitory computer readable medium embodied with programming instructions that when executed cause a processor to access blood glucose measurement values and insulin data from a memory. The processor may identify blood glucose measurement values that exceed an upper target blood glucose set point as high events and blood glucose measurement values that are below a lower target blood glucose set point as low events. High event patterns may be identified in the high events that overlap and low event patterns may be identified in the low events that overlap, where in the overlap is determined based on a window of time. A bolus event rule set may be applied to the identified overlapping high event patterns and identified overlapping low event patterns. Based on the application of the bolus event rule set to the respective identified overlapping low events and the respective identified low events, bolus events may be identified that correspond to respective identified high events and respective identified low events for analysis. The identified bolus events may be appended to a corresponding identified high event or a corresponding identified low event in the memory. A graphical user interface may be populated with a number of low event patterns with appended bolus events and with another, different number of high events with appended bolus events.

A system is disclosed that includes a memory and a data analysis processor. The memory may be operable to store a user's continuous blood glucose monitor data and data from a user's personal diabetes management device, wherein the user's continuous blood glucose monitor data includes blood glucose measurement values and the data from the user's personal diabetes management device includes insulin delivery data. The data analysis processor may include circuitry operable to execute programming code stored in the memory, and the data analysis processor may be operable to obtain blood glucose measurement values and insulin delivery data and the data from the user's personal diabetes management device for a predetermined time period. The data analysis processor may be operable to identify in the normalized data blood glucose measurement values that exceed an upper target blood glucose set point as high events and blood glucose measurement values that are less than a lower target blood glucose set point as low events. A high event overlap rule set may be applied to the high events and a low event overlap rule set to the low events by the data analysis processor. Based on the application of the high event overlap rule set to the high events and the low event overlap rule to the low events, the data analysis processor may identify overlapping high events and overlapping low events for analysis. the data analysis processor may identify as a high event pattern of the overlapping high events those high events that fall within a set high event overlap duration and day range and identify as a low event pattern of the overlapping low events those low events that fall within a set low event overlap duration and day range. A respective pattern weight may be applied by the data analysis processor to each identified high event and each identified low event based on criteria specific to high events and specific to low events. The data analysis processor may populate a graphical user interface with a number of high event patterns and with another, different number of low event patterns.

### BRIEF DESCRIPTION OF THE DRAWINGS

FIG. 1A illustrates an example of a wearable drug delivery system that is suitable for use with and in the implementation the subject matter described herein.

FIG. 1B illustrates an example system flow diagram for obtaining and processing data to be used by the according to the subject matter described herein.

FIG. 2A illustrates a flowchart of an example of a data visualization algorithm that determines a time of day pattern present in blood glucose blood measurement values and insulin values for a predetermined period of time according to the subject matter described herein.

FIG. 2B illustrates a flowchart of an example of a data visualization algorithm that determines highs and lows after a bolus present in blood glucose blood measurement values and insulin values for a predetermined period of time according to the subject matter described herein.

FIG. 3 illustrates an example of a graphical user interface for the presentation of a time in range of a user's blood glucose as well as low event patterns and high event patterns as determined by the data visualization algorithm according to the subject matter described herein.

FIG. 4 illustrates an example of a graphical user interface for the presentation of a trend of blood glucose measurement values as well as low event patterns and high event patterns representing when blood glucose measurement values were below a low target set point and above a high target set point according to the subject matter described herein.

FIG. 5A illustrates an example of a graphical user interface for presentation of details of a specific high blood glucose event according to the subject matter described herein.

FIG. 5B illustrates an example of the graphical user interface of FIG. 5A with a legend indicating mode and insulin usage of a drug delivery device according to the subject matter described herein.

FIG. 5C illustrates an example of the graphical user interface of FIG. 5A with a pop up window indicating bolus delivery time, bolus amount and estimated carbohydrates according to the subject matter described herein.

FIG. 5D illustrates an example of the graphical user interface that presents information related to multiple high event patterns that occurred in the evening with recommendations according to the subject matter described herein.

FIG. 6A illustrates an example of a graphical user interface for presentation of a specific low blood glucose event according to the subject matter described herein.

FIG. 6B illustrates an example of the graphical user interface of FIG. 6A with a legend indicating drug delivery device mode and insulin usage of a drug delivery device according to the subject matter described herein.

FIG. 6C illustrates an example of the graphical user interface of FIG. 6A with a pop up window indicating bolus delivery time, bolus amount and estimated carbohydrates according to the subject matter described herein.

FIG. 6D illustrates an example of the graphical user interface that presents information related to multiple high event patterns that occurred in the evening with recommendations according to the subject matter described herein.

FIG. 7A illustrates an example of a graphical user interface for presentation of a high event after a bolus according to the subject matter described herein.

FIG. 7B illustrates an example of the graphical user interface of FIG. 7A with a legend indicating drug delivery device mode and insulin usage of a drug delivery device according to the subject matter described herein.

FIG. 7C illustrates an example of the graphical user interface of FIG. 7A with a pop up window indicating bolus delivery time, bolus amount and estimated carbohydrates according to the subject matter described herein.

FIG. 8A illustrates an example of a graphical user interface for presentation of a low event after a bolus according to the subject matter described herein.

FIG. 8B illustrates an example of the graphical user interface of FIG. 8A with a legend indicating drug delivery device mode and insulin usage of a drug delivery device according to the subject matter described herein.

FIG. 8C illustrates an example of the graphical user interface of FIG. 8A with a pop up window indicating bolus delivery time, bolus amount and estimated carbohydrates according to the subject matter described herein.

FIG. 9A illustrates an example of the graphical user interface providing a daily summary over a period of time according to the subject matter described herein.

FIG. 9B illustrates a detailed graphical user interface view example of a day example with a legend from the daily summary graphical user interface example of FIG. 9A according to the subject matter described herein.

FIG. 10A illustrates an example of the graphical user interface providing a weekly summary according to the subject matter described herein.

FIG. 10B illustrates a detailed graphical user interface view example of a weekly time in range from the weekly summary graphical user interface example of FIG. 10A according to the subject matter described herein.

FIG. 10C illustrates a detailed graphical user interface view example of a weekly bolus and insulin summary from the weekly summary graphical user interface example of FIG. 10A according to the subject matter described herein.

FIGS. 11-24 illustrate ornamental features of examples of graphical user interfaces.

### DETAILED DESCRIPTION

A type of medication delivery algorithm (MDA) may include an “artificial pancreas” algorithm-based system, or more generally either an automatic insulin delivery (AID) application/algorithm or an artificial pancreas (AP) application. For ease of discussion, the computer programs and computer applications that implement the medication delivery algorithms or applications may be referred to herein as an “AID application.” An AID application or algorithm may be configured to provide automatic delivery of insulin based on an analyte sensor input, such as signals received from an analyte sensor, such as a continuous blood glucose monitor, or the like. The signals from the analyte sensor may contain blood glucose measurement values, timestamps, or the like.

In addition, or alternatively, while the disclosed examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AID application and algorithms may be operable to perform various functions related to open loop operations, such as the generation of prompts requesting the input of information such as diabetes type, weight or age. Similarly, a dosage amount of insulin may be received by the AID application or algorithm from a user via a user interface. Other open-loop actions may also be implemented by adjusting user settings or the like in an AID application or algorithm.

Systems, devices, computer readable media and methods in accordance with the present disclosure are now described more fully hereinafter with reference to the accompanying drawings, where one or more examples are shown. The systems, devices, and methods described herein may be embodied in many different forms and are not to be construed as being limited to the examples set forth herein. Instead, these examples are provided so the disclosure is thorough and complete, and may fully convey the scope of the techniques and devices to those skilled in the art. Each of the systems, devices, media and methods disclosed herein provides one or more advantages over conventional systems, components, and methods.

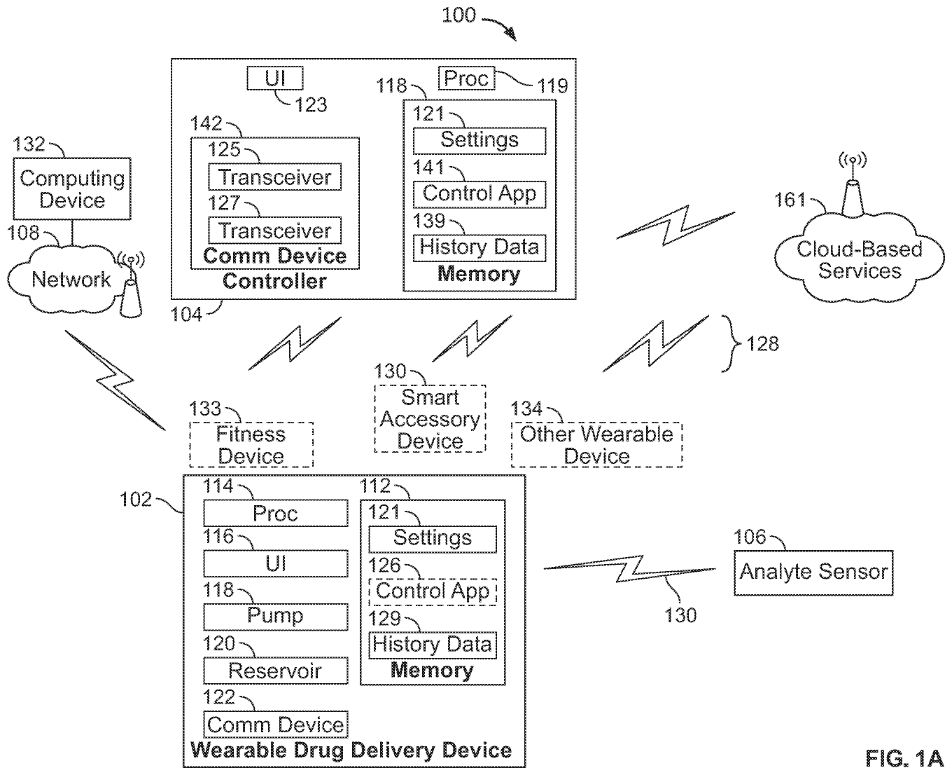

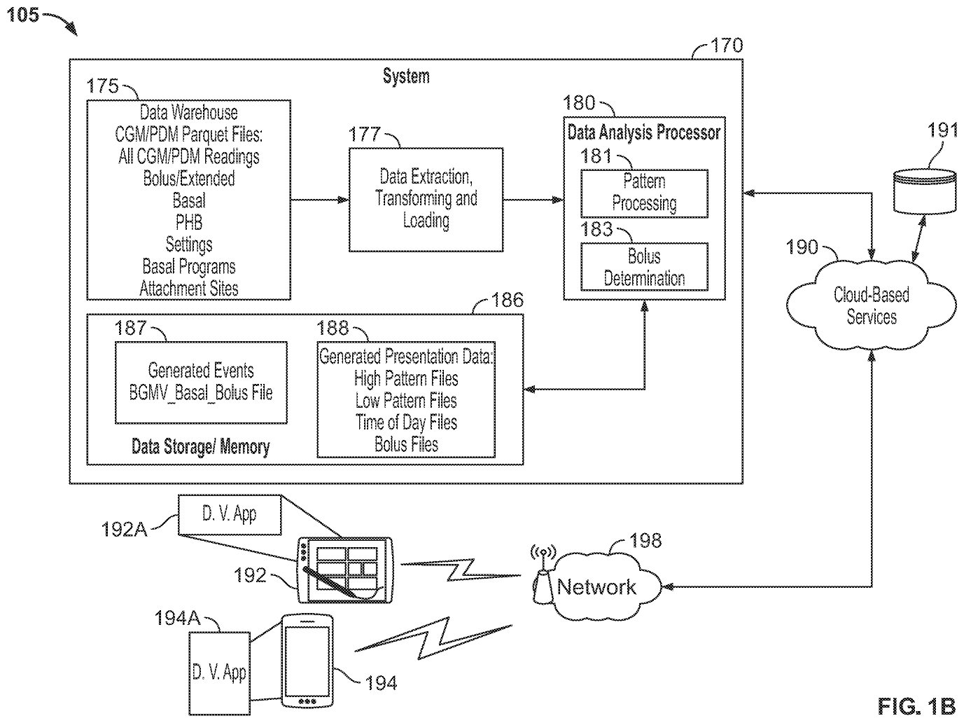

FIG. 1A illustrates a drug delivery system operable to be used with and to implement the examples as described herein.

In some examples, the drug delivery system 100 is suitable for delivering insulin to a user in accordance with the disclosed embodiments. The drug delivery system 100 may include a wearable drug delivery device 102, a controller 104 and an analyte sensor 106.

The wearable drug delivery device 102 may be a wearable device that is worn on the body of the user. The wearable drug delivery device 102 may be directly coupled to a user (e.g., directly attached to the skin of the user via an adhesive, or the like). In an example, a surface of the wearable drug delivery device 102 may include an adhesive to facilitate attachment to the skin of a user.

The analyte sensor 106 may be operable to collect physiological condition data such as blood glucose measurement values, and, in some examples, also provide a time stamp of when a respective blood glucose measurement was collected.

The wearable drug delivery device 102 may include a processor 114. The processor 114 may be implemented in hardware, software, or any combination thereof. The processor 114 may, for example, be a microprocessor, a logic circuit, a field programmable gate array (FPGA), an application specific integrated circuit (ASIC) or a microprocessor coupled to a memory. The processor 114 may maintain a date and time as well as be operable to perform other functions (e.g., calculations or the like). The memory 112 may include random access memory (RAM), read only memory (ROM), optical storage, magnetic storage, removable storage media, solid state storage or the like. The memory 112 may be operable to store settings 121, an optional control application 126 and history data 129. The processor 114 may be operable to execute the optional control application 126 stored in the memory 112 that enables the processor 114 to direct operation of the wearable drug delivery device 102. The control application 126 may be an AID algorithm that is operable to control insulin delivery to the user per an AID control approach as describe herein. In addition, the memory 112 may store settings 121 for a user, such as graphical user display preferences, healthcare provider communication settings, AID/MDA algorithm settings, control application settings and/or the like. The ADI and/or control application settings may include information and settings, such as maximum insulin delivery, insulin sensitivity settings, insulin correction factor settings, insulin-to-carbohydrate settings, total daily insulin (TDI) settings, and the like. The history data 129 may also include different data entries. In a first example, the history data 129 may have data entries that include insulin output history that may include at least basal insulin doses (i.e., an amount of insulin delivered in each basal insulin dose), a respective time for each of the basal insulin doses, bolus insulin doses (i.e., an amount of insulin delivered in each bolus insulin dose) and a respective time for each of the bolus insulin doses. In a second example, the history data 129 may have data entries that include insulin output history that may include at least basal insulin doses (i.e., an amount of insulin delivered in each basal insulin dose), a respective time for each of the basal insulin doses, bolus insulin doses (i.e., an amount of insulin delivered in each bolus insulin dose), a respective time for each of the bolus insulin doses as well as blood glucose measurement values (and corresponding timestamps of each respective measurement value) received from the analyte sensor 106.

The wearable drug delivery device 102 may include a reservoir 120. The reservoir 120 may be operable to store drugs, medications or therapeutic agents suitable for automated delivery, such as insulin, morphine, methadone, hormones, glucagon, glucagon-like peptide, blood pressure medicines, chemotherapy drugs, combinations of drugs, such as insulin and a glucagon-like peptide, or the like. So, while the description may refer primarily to insulin, the wearable drug delivery device 102 and controller 104 may be configured to output the above listed drugs, medications, therapeutic agents, or combinations thereof according to respective algorithms that may replace the above referenced AID or control algorithms/applications. A fluid path (not shown) from the reservoir 120 to the user may be provided via tubing coupled to a needle/cannula (not shown) that enables the outputted drug, medication, therapeutic agent or combination to be delivered subcutaneously or the like to a user. The wearable drug delivery device 102 may be operable, based on control signals from the processor 114, to expel or output the drugs, medications or therapeutic agents, such as insulin, from the reservoir 120 to deliver doses of the drugs, medications or therapeutic agents, to the user via the fluid path.

There may be one or more communications links 128 with one or more devices physically separated from the wearable drug delivery device 102 including, for example, a controller 104 of the user (or of a caregiver of the user) and/or a sensor 106. The communication links 128 may be any wired or wireless communication link that is formed or operating according to any known communications protocol or standard, such as Bluetooth®, Wi-Fi, a near-field communication standard, a cellular standard, or any other wireless protocol.

The wearable drug delivery device 102 may also include a user interface 116, such as an integrated display device for displaying information to the user, and in some embodiments, receiving information from the user. For example, the user interface 116 may include a touchscreen. Additionally, or alternatively, the user interface 116 may include one or more input devices, such as buttons, a knob or a keyboard that enable a user to provide an input.

In addition, the processor 114 may be operable to receive data or information from the analyte sensor 106 as well as other devices that may be operable to communicate with the wearable drug delivery device 102.

The wearable drug delivery device 102 may interface with a network 108. The network 108 may include a local area network (LAN), a wide area network (WAN) or a combination therein. A computing device 132 may be interfaced with the network, and the computing device may be operable to communicate with the wearable drug delivery device 102. In an example, the computing device 132 may be a healthcare provider device and be operable to communicate with a user's controller 104 and interact to obtain information, store and/or update settings of the wearable drug delivery device 102, the controller 104 and/or analyte sensor 106, and the like. For example, the computing device 132 may be operable to act as a backup device for the control application 141 or 126, or the like. The AID algorithm operating as, or in cooperation with, the control application 141 may present a graphical user interface on the computing device 132 enabling the input and presentation of information related to the AID algorithm.

The drug delivery system 100 may include an analyte sensor 106 for sensing the levels of one or more analytes of a user. The analyte levels may be used as physiological condition data and be sent to the controller 104 and/or the wearable drug delivery device 102. The sensor 106 may be coupled to the user by, for example, adhesive or the like and may provide information or data on one or more medical conditions and/or physical attributes of the user. The sensor 106 may be a continuous glucose monitor (CGM), or another type of device or sensor that provides that is operable to provide blood glucose concentration measurements. In addition, or alternatively, to blood glucose measurements, the sensor 106 may be operable to provide other metabolic or blood-related measurements. The sensor 106 may be physically separate from the wearable drug delivery device 102 or may be an integrated component thereof. The sensor 106 may provide the processor 114 and/or processor 119 with physiological condition data indicative of measured or detected blood glucose levels of the user. The information or data provided by the sensor 106 may be used in calculating summary data reports, statistical analysis, the presentation of the results of the summary data reports and statistical analysis as well as being operable to calculate and implement adjustments to drug delivery operations of the wearable drug delivery device 102.

In the example, the controller 104 of the drug delivery system 100 may include a processor 119 and a memory 118. The controller 104 may be a special purpose device, such as a dedicated personal diabetes manager (PDM) device. The controller 104 may be a programmed general-purpose device that is a portable electronic device, such as any portable electronic device, smartphone, smartwatch, fitness device, tablet or the like including, for example, a dedicated processor, such as processor, a micro-processor or the like. The controller 104 may be used to program or adjust operation of the wearable drug delivery device 102 and/or the sensor 106. The processor 119 may execute processes to manage a user's blood glucose levels and to control the delivery of the drug or therapeutic agent from the wearable drug delivery device 102. The processor 119 may also be operable to execute programming code stored in the memory 118. For example, the memory 118 may be operable to store a control application 141, such as an AID algorithm for execution by the processor 119. The control application 141 may be responsible for controlling the wearable drug delivery device 102, including the automatic delivery of insulin based on recommendations and instructions from the AID algorithm, such as those recommendations and instructions described herein.

The memory 118 may store the one or more control application(s) 141, settings 121 for the insulin delivery device 102 and history data 139 like those described above. The memory 118 may be operable to store other data and/or programs. In a first example, the history data 139 may include insulin output history that may include at least basal insulin doses (i.e., an amount of insulin delivered in each basal insulin dose), a respective time for each of the basal insulin doses, bolus insulin doses (i.e., an amount of insulin delivered in each bolus insulin dose) and a respective time for each of the bolus insulin doses. In a second example, the history data 139 may include insulin output history that may include at least basal insulin doses (i.e., an amount of insulin delivered in each basal insulin dose), a respective time for each of the basal insulin doses, bolus insulin doses (i.e., an amount of insulin delivered in each bolus insulin dose), a respective time for each of the bolus insulin doses as well as blood glucose measurement values (and corresponding timestamps of each respective measurement value) received from the analyte sensor 106.

The controller 104 may include a user interface (UI) 123 for communicating with the user. The user interface 123 may include a display, such as a touchscreen, for displaying information. The touchscreen may also be used to receive input when it is a touch screen. The user interface 123 may also include input elements, such as a keyboard, button, knob or the like. In an operation example, the user interface 123 may include a touchscreen display controllable by the processor 119 and be operable to present the graphical user interface, and in response to the received input, the touchscreen display is operable to generate a signal indicative of the subjective insulin need parameter. In an operational example, the user interface may be a touchscreen display controllable by the processor and operable to present a graphical user interface. The touchscreen display, under control of the processor 119, may be operable to, in response to the received input, generate a signal indicative of the subjective insulin need parameter as described with reference to earlier examples.

The controller 104 may interface via a wireless communication link of the wireless communication links 128 with a network, such as a LAN or WAN or combination of such networks that provides one or more servers or cloud-based services 161 via communication device 142. The communication device 142, which may include transceivers 127 and 125, may be coupled to the processor 119. The communication device 142 may be operable to transmit communication signals (e.g., command and control signals) to and receive communication signals (e.g., via transceivers 127 or 125) from the wearable drug delivery device 102 and the analyte sensor 106. In an example, the communication device 142 may include a first transceiver, such as 125 may be a Bluetooth transceiver that is operable to communicate with the communication device 142 of the wearable drug delivery device 102, and a second transceiver, such as 127, that is a cellular or Wi-Fi transceiver operable to communicate via the network 108 with computing device 132 or with cloud-based services 110.

The cloud-based services 161 may be operable to store user history information, such as blood glucose measurement values over a set period of time (e.g., days, months, years), insulin delivery amounts (both basal and bolus dosages), insulin delivery times, types of insulin, indicated meal times, blood glucose measurement value trends or excursions or other user-related diabetes treatment information, specific factor settings including default settings, present settings and past settings, or the like.

Other devices, like smart accessory device 130 (e.g., a smartwatch or the like), fitness device 133 and other wearable device 134 may be part of the drug delivery system 100. These devices may communicate with the wearable drug delivery device 102 to receive information and/or issue commands to the wearable drug delivery device 102. These devices 130, 133 and 134 may execute computer programming instructions to perform some of the control functions otherwise performed by processor 114 or processor 119. These devices 130, 133 and 134 may include user interfaces, such as touchscreen displays for displaying information such as current blood glucose level, insulin on board, insulin deliver history, or other parameters or treatment-related information and/or receiving inputs, such as those described with reference to the examples of FIGS. 1-3. The display may, for example, be operable to present a graphical user interface for providing input, such as request a change in basal insulin dosage or delivery of a bolus of insulin. These devices 130, 133 and 134 may also have wireless communication connections with the sensor 106 to directly receive blood glucose level data or receive in parallel a presentation of the graphical user interface as shown in FIG. 1.

In another operational example, the controller 104 may be operable to execute programming code that causes the processor 119 of the controller 104 to perform the following functions. The processor 119 of the controller 104 may execute an AID algorithm that is one of the control applications 120 stored in the memory or memory 118. The processor may be operable to provide a graphical user interface on which is presented data as described with reference to the later examples.

When an input is received on the user interface 123 in response to presentation of the graphical user interface, the processor 119 may be operable to cause presentation of different event pattern information windows, drug treatment summary pages that include tables and/or information graphics that provide a visual indication and information in a visual format that is different than a listing of values. The graphical user interfaces as discussed herein provide information enhanced by data visualization techniques.

The processor 119 is also operable to collect physiological condition data related to the user from sensors, such as the analyte sensor 106 or a heart rate monitor of the fitness device 133 or the smart accessory device 130. In an example, the processor 119 executing the AID algorithm may determine a dosage of insulin to be delivered based on the collected physiological condition of the user and a specific factor determined based on the subjective insulin need parameter. The processor 119 may output a control signal via one of the transceivers 125 or 127 to the wearable drug delivery device 102. The outputted signal may cause the processor 114 to deliver command signals to the pump 118 to deliver an amount of insulin related to the determined dosage of insulin in the reservoir 120 to the user based on an output of the AID algorithm implemented by the control app 141. These signals related to the determined dosages and the like may be stored as history data (129, 139) in memories 112 and/or 118.

FIG. 1B illustrates an example system and flow diagram for analyzing data to generate data visualization graphical user interfaces according to the examples described herein.

An information environment 105 may include a number of components, such as a system 170, cloud based services 190, a network 198 and user devices 192 and 194. The system 170 may include a data repository 175, a data extraction, transformation and loading component 177, a data analysis processor 180 and a data storage/memory 186. The data repository 175 may be operable to store continuous glucose monitors/personal diabetes management device parquet files. The CGM/PDM parquet files may store all received CGM/PDM readings, Bolus/extended bolus dosage amounts, basal dosage amounts, patient history buffer (PHB) measurements, AID algorithm settings including target blood glucose setpoints and the like, basal programs and attachment site information.

The memory 186 may be operable to store a user's continuous blood glucose monitor data and data from the user's personal diabetes management device. Data extraction, transformation and loading circuitry 177 may be coupled to the memory 186 operable to normalize data for evaluation and analysis by the data analysis processor 180. The data analysis processor 180 may include a combination of circuitry and software. The data analysis processor 180 may include pattern processing 181 and bolus determination 183. The pattern processing 181 may include processing circuitry and/or programming code or instructions that when executed by the processing circuitry may be operable to identify patterns in the user's continuous blood glucose monitor data and data from the user's personal diabetes management device.

The bolus determination 183 may be programming code or instructions that when executed by processing circuitry may be operable to utilize the data from the user's personal diabetes management device to further analyze the user's continuous blood glucose monitor data to identify further patterns related to both the data from the user's personal diabetes management device and the user's continuous blood glucose monitor data.

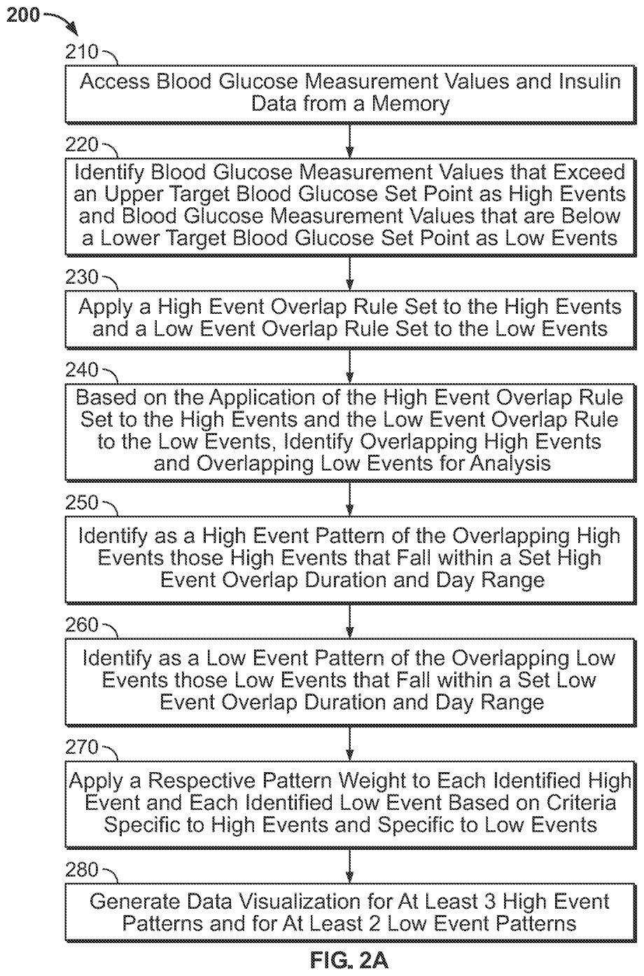

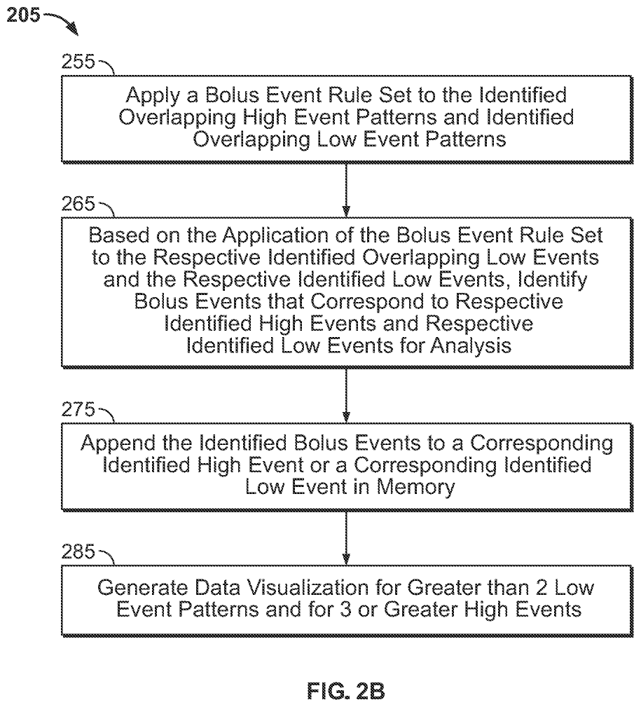

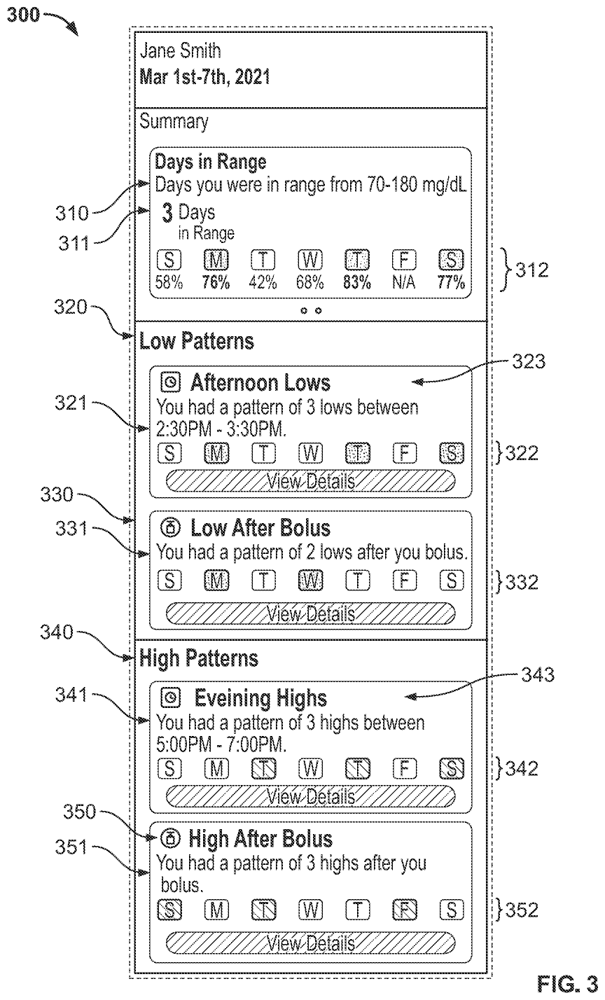

In a specific example, the pattern processing 181 and bolus determination 183 of the data analysis processor 180 may be implemented with a collection of Databricks' notebooks or the like that are operable to generate high and low event patterns on a scheduled basis. A notebook, for example, may be a web-based interface to a document that contains runnable code, visualizations, and/or narrative text. In an example a separate notebook may be established for each of the data visualizations that are to be presented in a graphical user interface described in more detail with reference to FIGS. 2A-10C. In the weekly summaries, also referred to as insights, the data visualization may be generated by reading data from last Sunday (beginning of day) to current Saturday (end of day), the generation runs on schedule on 1 AM EST or the like. A scheduler executed by the data visualization application may trigger different notebooks that are operable to generate the generated presentation files 188, such as the high pattern files, the low pattern files, the time of day files and the bolus files.

The patterns may be used to identify events that may be helpful to a user to better understand and implement their diabetes treatment plan. Based on the analysis performed by the data analysis processor 180, the identified patterns and events may be stored in the data storage/memory 186. For example, the data storage/memory 186 may include generated events, blood glucose measurement values, basal delivery data, and bolus deliver files and generated presentation data 188, such as high pattern files, low pattern files, time of day files and bolus files that may be presented via a data visualization application on a user device.

Alternatively, or in addition, the memory 186 may be populated the generated presentation files 188 and generated events 187 that are also, or alternatively, persisted on storage 191 coupled to the cloud-based services 190. A message may be sent to cloud-based services 190, such a cloud platform messaging service or the like, regarding the completion of the presentation files 188. This arrangement makes the persisted presentation files 188 and generated events 187 available for downstream systems to process and users to consume.

The data visualization application may be a computer application that enables the presentation data 188 and generated events 187 to be presented on a user device, such as a tablet device 192 or a smart phone 194. The smart phone 194 may include components and the functionality of controller 104 of FIG. 1 as described above. For example, the tablet device 192 may host an instance of a data visualization application 192A and the smart phone 194 may also host another instance of a data visualization application 194A.

Time of Day Patterns Algorithm

FIG. 2A illustrates a flowchart of an example of an algorithm that determines a time of day pattern present in blood glucose blood measurement values and insulin values for a predetermined period of time according to the subject matter described herein.

The algorithm presented in FIG. 2A may be referred to as an algorithm that detects a time of the day pattern. The algorithm 200 is operable to find reoccurring High or Low blood glucose patterns during a week (i.e., 168 hours). For example, at step 210, a processor executing programming code that implements the algorithm 200 may be operable to access blood glucose measurement values and insulin data from a memory. In an example, the memory by be operable to store at least one week of blood glucose measurement values (and a corresponding timestamp) and at least one week of doses of insulin output by a drug delivery device (and a corresponding insulin output timestamp). The doses of insulin include both basal doses and bolus doses as well as the timestamp. For example, the processor may be operable to perform a data extraction operation by obtaining the weekly blood glucose measurement values and insulin data. The processor may also obtain from the memory, such as from user settings, an upper (i.e., high) target blood glucose set point (e.g., 180 mg/dL) and a lower (i.e., low) target blood glucose set point (e.g., 70 mg/dL) for the user.

In addition, the processor may perform a data preparation process in which the processor cleans the data by considering blood glucose measurement values between 39-401 mg/dL and prepares a date and time field for different rows. For a limited data visualization mode, blood glucose measurement values outside this range (and related data, such as dates, times or the like) may also be considered just for output charts but not for algorithm. In some examples of the data, the date/time fields along with UtcOffset fields (e.g., coordinated universal time offsets for respective time zones) are present in the history data set obtained from the memory.

In addition, the history data may only be considered if time zone change frequency (i.e., how often the user changes time zones) is less than (<) 2 (i.e., happening only once a week) and for changes happening only once a week, the change is expected to be less than or equal to (<=) 1 hour. The algorithm 200 causes the processor when evaluating the blood glucose measurement values to filter out respective high events or low events with time zone changes inside the respective high or low event and 2 hours (or 3 hours for High/Low After Bolus) time slot before the respective high or low event and within a 1 hour time slot after a respective high or low event regardless of duration.

The algorithm 200 causes the processor to look for high and low events in the accessed blood glucose measurement values of the history data. For example, at step 220, the processor while executing algorithm 200 may be operable to identify blood glucose measurement values that exceed an upper target blood glucose set point as high events and blood glucose measurement values that are below a lower target blood glucose set point as low events. The processor may be operable to obtain the user's upper target blood glucose set point and lower target blood glucose set point from user settings stored in memory.

In more detail, when identifying high events and low events, the processor may locate start and end times of high and low events by identifying logged blood glucose measurement values from a data structure containing history data and by figuring out when the respective blood glucose measurement value crossed the respective upper target blood glucose set point and return back to the clinically normal range (e.g., 70-180 mg/dL or the like). For low events, the processor may consider a person to be above the low target blood glucose measurement set point when next 3-6 consecutive readings of blood glucose measurement values are greater than the low target blood glucose measurement set point; otherwise, the processor may consider that the user's blood glucose measurement values continue to be below the low target blood glucose measurement set point if the readings fluctuate across the low target blood glucose measurement set point.
The identified events may be stored in memory and/or be persisted on cloud storage, such as 161 of FIG. 1.

The identified high events may be identified in a previous week of history data and the processor evaluates, at 230, the identified high events to locate any overlapping time between the respective identified high events and based on any overlapping times being located, generates corresponding high event patterns. For example, the algorithm 200 may apply a high event overlap rule set that has high event criteria that define the degree of overlap in duration of overlapping time and the number of days. In addition, the algorithm 200 may apply a low event overlap rule set that has low event criteria that define the degree of overlap in duration of overlapping time and the number of days. The high event overlap rules may be different from the low event overlap rules. Examples of such high event overlap rules may include evaluating high events that overlap (>=30 min or <=6 hours as a high event duration rule) across three days (i.e., a number of overlapping days) and evaluating low events that overlap (>=15 min or <=6 hours as a low event duration rule) across two days (i.e., a number of overlapping days). Events can cross day boundaries, such as from Monday to or through Tuesday, Friday through to Sunday, and the like.

At 240, based on the application of the high event overlap rule set to the high events and the low event overlap rule to the low events, identify overlapping high events and overlapping low events for analysis. The analysis may occur at steps 250 and 260, the order of which is not particularly relevant.

At step 250, the processor may identify as a high event pattern of the overlapping high events of those high events that fall within a set high event overlap duration and day range.

For example, when identifying a high event pattern, the processor may calculate the average of the blood glucose measurement values for all of the identified high events by summing the estimated blood glucose measurement values during the respective high event duration and dividing the sum by the total number of identified high events during a window of time encompassing the respective high event duration (first time window and/or number of days).

At step 260, the processor may identify as a low event pattern of the overlapping low events of those low events that fall within a set low event overlap duration and day range. Similarly, when identifying a low event pattern, the processor may calculate the average of the blood glucose measurement values for all of the low events may be calculated by summing the estimated blood glucose measurement values during the respective low event duration and dividing the sum by the total number of low events during a window of time encompassing the respective low event duration (second time window and/or number of days). A first window of time may be used to identify each respective low event pattern.

In an example, the respective high and low patterns may be identified through the following more detailed steps:
1) when the high and low events have been identified, a next step for the identified high events may be to load the persisted high events (i.e., previously identified high events now stored in memory and/or cloud storage) and find the overlapping time between them. Similarly, the processor may for the identified low events, load the persisted (i.e., previously identified) low events and find the overlapping time between them by cross joining the different events (i.e., the identified high events, the persisted high events, the identified low events, and the persisted low events) intersecting time ranges may be found.
2) in response to finding intersecting time ranges and establishing the high event and low event patterns, an overlapping start time and end time is calculated and updated for each of the established patterns.
3) after the respective pattern start times and end times have been updated, any events that do not fit within the 30 minute overlap are removed and overlap times are recalculated based on the remaining events.
4) in a cleaning process, patterns that have a 30 minute overlap but that have only a single event are dropped as patterns because they no longer constitute a pattern.
5) after all patterns are cleaned, any overlapping patterns are merged if merging such patterns still maintains an overlap time of 30 minutes.
6) the merged patterns may be evaluated for intersecting time ranges and a reduceByKey operation or similar operation is performed by the processor to find the count of these intersecting time ranges. For identified high event patterns, the count should be greater than 3, and for the identified low event patterns, the count should be greater than 2.
7) After generating a table data structure using the identified high event patterns and the identified low event patterns, a pattern weight is assigned to each pattern as discussed in more detail below.

The algorithm 200 may generate additional insights based on the remaining events for use in providing recommendations or suggests as described with reference to other examples.

At 270, the processor may apply a respective pattern weight to each identified high event and each identified low event based on criteria specific to high events and specific to low events. Examples of pattern weights include pattern weight=0.50*duration of overlapping time+. 0.50*number of days that a particular high event pattern or low event pattern was found. The pattern weight for a respective high event or low event pattern may be appended in a column corresponding to the respective high event or low event pattern. After appending the weight column to different high event and low event patterns identified, the tables may be converted to j son files or the like and saved on a cloud storage platform.

At 280, the processor may generate in graphical user interface a data visualization for presentation that includes at least 3 high event patterns and at least 2 low event patterns. In addition, a queue message may be generated to notify the downstream processes that algorithm data is available now for consumption. Examples and discussion of the improved data visualization are provided with reference to later figures.

FIG. 2B illustrates a flowchart of an example of an algorithm 205 that determines highs and lows after a bolus present in blood glucose blood measurement values and insulin values for a predetermined period of time according to the subject matter described herein.

The high/low after bolus algorithm 205 may be performed based on results provided by the algorithm 200 as described with reference to and as shown in FIG. 2B. The algorithm 205 is operable to find high and low events occurring after a bolus event during a week. Each identified high event or low event can have multiple boluses preceding them as long as the boluses fall within a bolus identification window. A bolus identification window is a per (Event start time—30 mins to Event start time—3 hours)

Data will only be considered if time zone change frequency is <2 (happening only once a week) and for change happening only once a week it should be <=1 hour. Algorithm will filter out events with time zone change inside event and a 2 hour event time slot before an event and 1 hour time slot after event (either high or low) irrespective of duration of the event, or in the aggregate or 3 hour window to identify a High/Low event After Bolus event.

A processor may execute steps 210-260, or even 270 prior to beginning execution of algorithm 205.

The processor when executing algorithm 205 may, at 255, apply a bolus event rule set to the identified overlapping high event patterns and identified overlapping low event patterns. The bolus event rule set may have criteria for the identified overlapping high event patterns and identified overlapping low event patterns. For example, the processor may apply a rule that determines whether any bolus event occurred that preceded a respective identified high or low event pattern between a defined window of time, such as t minus 3 hours or t minus 15 minutes, where t is a start time of the identified high event or the identified low event. The defined window of time may be the same or different for the identified high event pattern or identified low event pattern. If a bolus (any bolus: correction, manual, automatic, or extended) is determined to have occurred within the defined window of time for the identified high event pattern or an identified low event pattern, then the bolus corresponds the identified high event pattern or an identified low event pattern.

At 265, the processor may be operable to identify bolus events that correspond to respective identified high events and respective identified low events for analysis based on the application of the bolus event rule set to the respective identified overlapping low events and the respective identified low events.

At 275, the processor may append the identified bolus events to a corresponding identified high event or a corresponding identified low event in memory. The final bolus low and bolus high patterns table may be converted, for example, to j son files and saved to a cloud storage platform. Queue messages may be generated to notify downstream processes that algorithm data is available now for consumption. Downstream processes may, for example, be a backend application programming interface (API) service that may obtain the generated file based on the message. The backend API may process the file and save it into a database. A web portal may be operable to show the data based on the information saved in the database.

The processor, at 285, may generate data visualization for greater than 2 low event patterns and for 3 or greater high events.

FIG. 3 illustrates an example of a graphical user interface for the presentation of a time in range of a user's blood glucose as well as low event patterns and high event patterns representing when time in range was below a low target set point and above a high target set point according to the subject matter described herein.

The graphical user interface 300 is a weekly summary of a number of days in range 310 for the user, Jane Smith. The days of the week are from Sunday to Saturday and represented using the first letter of each day (i.e., Sunday (S)—the first S, Monday as M, Tuesday as T, and so on. The summary 310 presents the user's low blood glucose target set point (i.e., 70 mg/dL) and the user's high blood glucose target set point (i.e., 180 mg/dL). Time in range is a time measurement of how much time the user's blood glucose stayed between the user's low blood glucose target set point and the user's high blood glucose target set point. The time in range is presented as a percentage of the 24 hour time period that constitutes a day. The graphical user interface 300 shows the number of total “days in range” 311, in this case, 3, that the user was in range. A weekly legend 312 shows the percentage of time during specific days that the user was within range of their high and low blood glucose target set points.

The weekly summary graphical user interface 300 also shows the identified low patterns 320. Low pattern details are shown below the heading “Afternoon Low” 323. The low pattern details 321 show what time during the days of the week that the low pattern was identified and the low pattern legend 322 shows on which days that the low patterns were found. The heading “Afternoon Low” 323 and the low pattern details 321 may be one of four different time frames: Morning (e.g., 5 am-12 pm), Afternoon (e.g., 12 pm-4 pm), Evening (e.g., 4 pm-10 pm) and Night (e.g., 10 pm-5 am). In some instances, the time frames may be fixed at the listed default time frames. In other examples, the time frames may be set in a user preference setting or the like that are part of the application.

Under the low patterns heading 320, the graphical user interface 300 also presents the low event after bolus patterns 330. The low event after bolus patterns 330 also includes a low event after bolus details 331 that indicate the number of low events after bolus patterns that occurred during the week of the summary. The low after bolus pattern legend 332 shows on which days that the low after bolus patterns were found, which in this case were Monday (M) and Wednesday (W).

Under the high patterns heading 340 are evening highs label 343. Below the evening highs label 343 are presented in the summary graphical user interface 300, the high pattern details 341. The high pattern details 341 may indicate for example a time window during which the high patterns were identified. In the illustrated example, the time window is “5:00 pm to 7:00 pm.” Of course, the time window can have different times, such as 4 pm-6 pm, 6:15 pm-8:30 pm or the like. The high event pattern legend 342 shows on which days that the high event patterns were found, which in this case were Tuesday (T), Thursday (2^{nd} T) and Saturday (2^{nd} S)

Also presented in the summary graphical user interface 300 is the high event after bolus patterns 350. The high event after bolus patterns 350 also includes a high event after bolus details 351 that indicate the number of high events after bolus patterns that occurred during the week of the summary. The high after bolus pattern legend 352 shows on which days that the low after bolus patterns were found, which in this case were Sunday (1^{st} S), Tuesday (T), and Wednesday (W).

Of course, more or less information may be shown in the weekly summary presented in the graphical user interface 300. For example, each of the “Afternoon Lows” (323), “Low after Bolus” (330), “Evening Highs” (343), and “High after Bolus” (350) have a “view details” soft button that may be selected to present the details of the respective events.

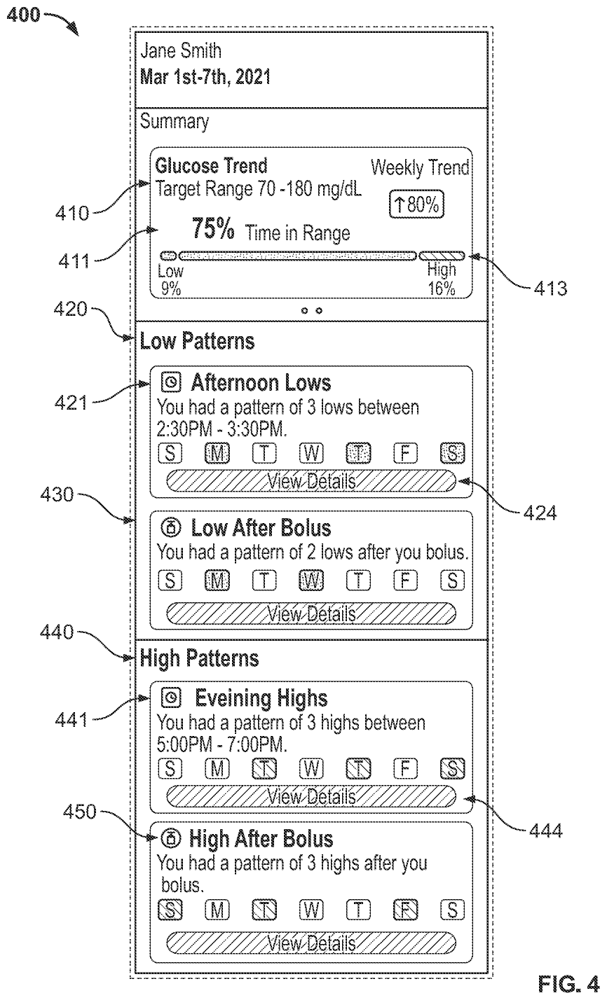

FIG. 4 illustrates an example of a graphical user interface for the presentation of a trend of blood glucose measurement values as well as low event patterns and high event patterns. The low event patterns and high event patterns represent when blood glucose measurement values were below a low target set point and above a high target set point according to the subject matter described herein.

The graphical user interface 400 is a weekly summary of a user's glucose trend 410 for the user, Jane Smith, during the same week as that presented in graphical user interface 300 of FIG. 3. The glucose trend summary 410 indicates that the weekly trend is upward (represented by the up arrow) by 8% which may be a percentage compared to the glucose measurements from the previous week, for example. The glucose trend details 411 indicates an overall percentage of time for the summarized week that the user's blood glucose measurement values were within range, which in this specific example, is 75% of the blood glucose measurement values obtained for the summarized weeks. In addition, the glucose trend summary 410 may include a range line that identifies a percentage of the blood glucose measurement values collected during the summarized week that were below the low target blood glucose measurement set point (in this example, 9%) as well as a percentage of the blood glucose measurement values that were above the high target blood glucose measurement set point (in this example, 16%). In the example, the low target blood glucose measurement set point is 70 mg/dL and the high target blood glucose measurement set point is 180 mg/dL. Of course, these values may be changed according to respective user preferences or diabetes treatment plans.

The graphical user interface 400 shows the low patterns 420 and high patterns 440 below the glucose trend summary 410. The low patterns 420 include the afternoon lows 421 and low after bolus 430 that are the same as those described with reference to the afternoon lows 323 and low after bolus 330 of FIG. 3. Similarly, the high patterns 440 include the evening highs 441 and high after bolus 450 that are the same as those described with reference to the evening highs 343 and high after bolus 350 of FIG. 3. Therefore, a detailed description of these items is not presented with respect to FIG. 4.

Of course, more or less information may be shown in the weekly summary presented in the graphical user interface 400. For example, below or around each of the “Afternoon Lows” (421), “Low after Bolus” (430), “Evening Highs” (441), and “High after Bolus” (450) have a “view details” soft button, such as 445, that may be selected to present the details of the respective events.

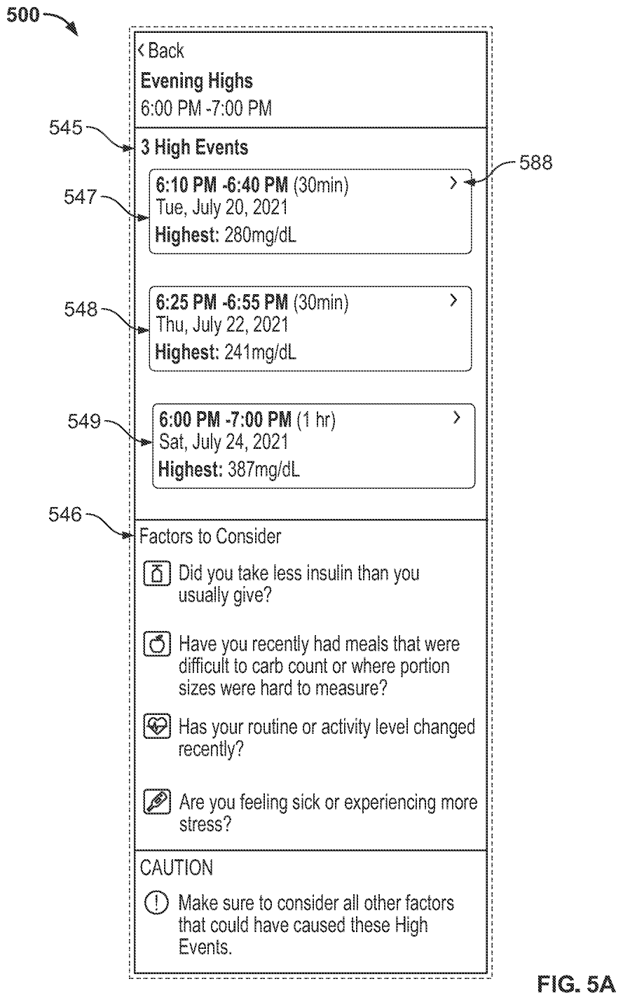

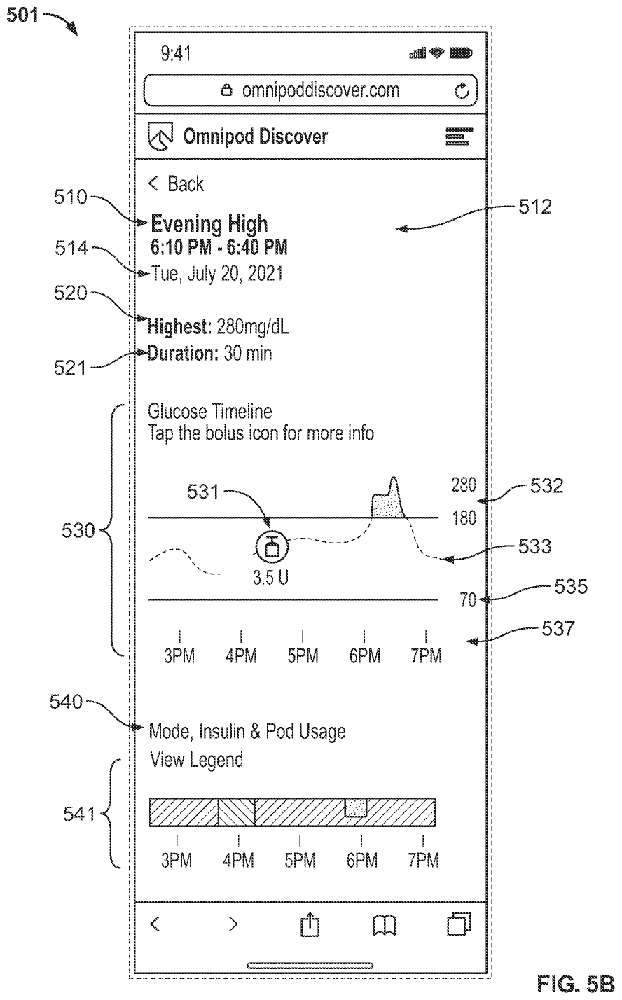

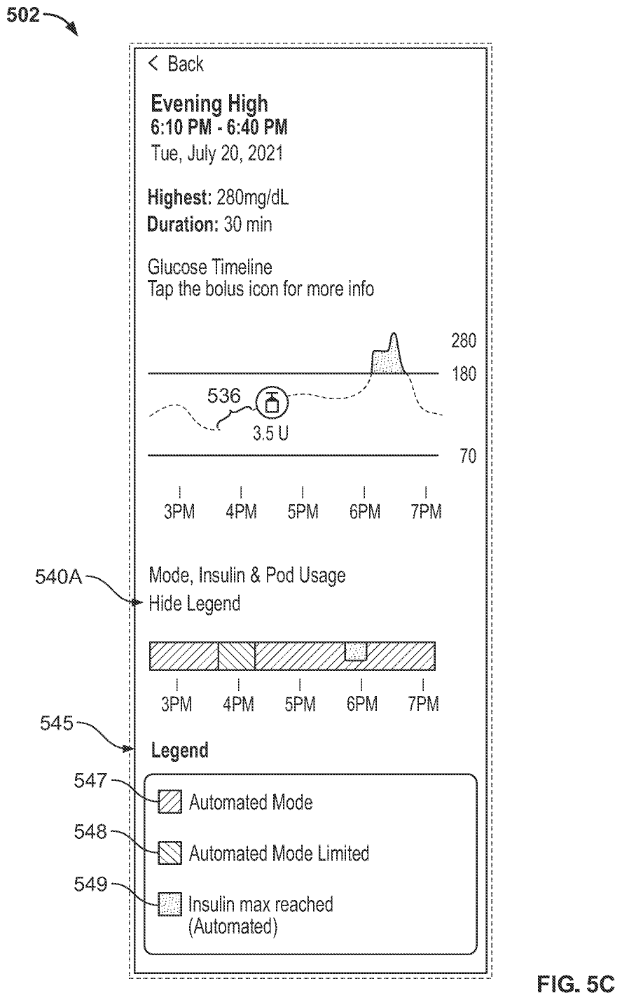

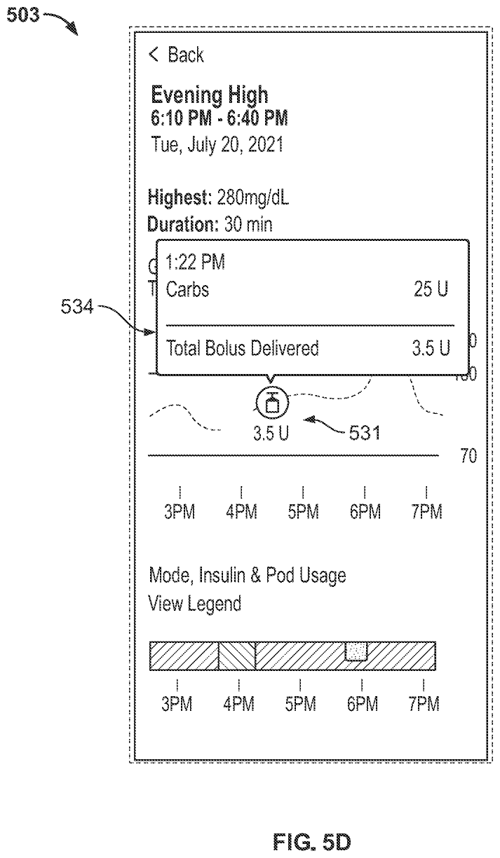

FIG. 5A illustrates an example of the graphical user interface that presents information related to multiple high event patterns that occurred in the evening with recommendations according to the subject matter described herein.

To access the information related to multiple high event patterns that occurred in the evening with recommendations, a user may select the “View Details” button 344 beneath the high event pattern legend 342 of the evening highs 343 of FIG. 3 or the “View Details” button 444 beneath the high event pattern legend of the evening highs 441. In response to the selection, details of the multiple high event patterns (e.g., “Evening Highs”) are presented in the graphical user interface 500. The graphical user interface 500 presents the details of each of the 3 high event patterns 545 in a respective high event window 547, 548 and 549. While the number 3 is shown for the number of high event patterns 545, the number shown may be greater than 3 if the number of identified high event patterns is greater and/or based on user settings (i.e., 3 is a minimum default but the user can set the number of high events to be presented to greater than 3). The high event window 547 shows the details of the Tuesday high event pattern, the high event window 548 shows the details of the Thursday high event pattern and the high event window 549 shows the details of the Saturday high event pattern. In the example, the details in each high event window 547-549 may include a highest blood glucose measurement value obtained during the high event, a duration of the high event, such as 30 minutes, 45 minutes, 1 hour or the like, as well as a user interface operator, such as 588, that enables presentation of further details regarding the specific high event pattern.

In addition, the graphical user interface 500 presents recommendations or “Factors to Consider” 546 based on the respective high event patterns. The recommendations 546 may be based on the highest blood glucose measurement value obtained during the high event, the duration of the high event and/or other information.

FIG. 5B illustrates an example of a graphical user interface for presentation of details of a specific high blood glucose event according to the subject matter described herein.

In response to selection of user interface operator 588 of FIG. 5A or selection of a “view details” soft button such as one associated with Evening Highs, such as 441 of FIG. 4, the graphical user interface 501 of FIG. 5B may be presented to show details of an evening high. In the example graphical user interface 501, the “Evening High” heading 510 may be presented. Below the “Evening High” heading 510 may be presented a time window 512 and a high event date 514 of a high event that satisfied criteria for a high event for this particular user. The graphical user interface 501 may also include the highest blood glucose measurement value that is measured during the time window 512 of the high event and the duration 521 of that highest blood glucose measurement value.

Also presented in the graphical user interface 501 is a glucose timeline 530. The glucose timeline 530 is a graphic with a curve 533 showing a user's blood glucose measurement values over a timeline 537 which, in this example, is approximately 2:30 pm through to a short time after 7:00 pm, like 7:05 pm or so. The glucose timeline 530 also has borders that represent the high target blood glucose set point border 532 (in this example, 180 mg/dL) and the low target blood glucose set point border 535 (in this example, 70 mg/dL) and a bolus indicator 531. The bolus indicator 531 also includes a label showing the number of units of insulin that have been delivered. In the example, the bolus indicator 531 indicates that a bolus of 3.5 units (U) was delivered at approximately 4:30-4:40 pm. Also, the graphical user interface 501 includes a color-coded timeline mode indicator 541 that indicates modes of the drug delivery system during the timeline 537. The modes are color-coded but a legend may be presented by selection of the “View Legend” soft button 540.

FIG. 5C illustrates an example of the graphical user interface of FIG. 5B with a legend indicating mode and insulin usage of a drug delivery device according to the subject matter described herein. The graphical user interface 502 of FIG. 5C includes elements from the graphical user interface 501 of FIG. 5B that function in the same manner as described with reference to FIG. 5B so a detailed discussion of those elements is not repeated.

In response to the selection of the “View Legend” soft button 540, the graphical user interface 502 may present the legend 545 beneath soft button 540A that now presents “Hide Legend.” The legend 545 may present different modes that are color-coded such as an automated mode 547 and an automated mode limited 548. In addition, or alternatively, the legend 545 may indicate when thresholds (both positive and negative thresholds) are reached or exceeded, such as in the case of “Insulin max reached (automated)” 549.

The automated mode limited 548 color coded label that traverses approximately 3:45 pm-4:15 pm on the color-coded timeline mode indicator 541 of FIG. 5B. In an example, the data visualization algorithm described with reference to other examples may be operable to identify when blood glucose measurement values are missing during a time period, such as in gap 536. As mentioned above with reference to FIG. 1, blood glucose measurement values may be received from a continuous blood glucose monitor (CGM) at different time intervals during operation of the wearable drug delivery device. For example, some CGMs may output signals containing blood glucose measurement values every 5 minutes or even less, such as every minute. There are a number of different reasons why the data visualization application may not present a blood glucose measurement value at a particular time. For example, a CGM may fail to output a signal due to a faulty reading or loss of connectivity, the controller may not receive the signal from the CGM due to interference or is out of range, the signal is corrupted by interference or the like. Alternatively, the data visualization application (shown and described in another example) may receive a subsequent blood glucose measurement value that is so much higher or lower than the immediately preceding blood glucose measurement that it is considered an outlier and is not used. Whatever the reason for not receiving or using the blood glucose measurement value at the expected time, the data visualization algorithm is operable to indicate the missing blood glucose measurement value as a gap 536 in the curve 533 and also indicate in the color-coded timeline that the AID algorithm entered the automated mode limited mode.

FIG. 5D illustrates an example of the graphical user interface of FIG. 5B with a pop up window indicating bolus delivery time, bolus amount and estimated carbohydrates according to the subject matter described herein.

The graphical user interface 503 includes the bolus indicator 531 that has a label showing the number of units of insulin (i.e., 3.5 U) that have been delivered at approximately 4:30-4:40 pm. In response to selection of the bolus indicator 531, a pop-up window 534 may be presented that indicates a time when the bolus was delivered (e.g., approximately 4:30 pm), a number of carbohydrates provided to a bolus calculator of the AID algorithm (e.g., 25 U) and an amount of a total bolus dosage (e.g., 3.5 U) that was delivered to offset the carbohydrates. The pop-up window 534 may be presented for a predetermined period of time (e.g., 30 seconds, 1 minute, 3 minutes or the like). Alternatively, or in addition, the pop-up window 534 may be presented until the graphical user interface 503 is interacted with subsequent to the selection of the bolus indicator 531.

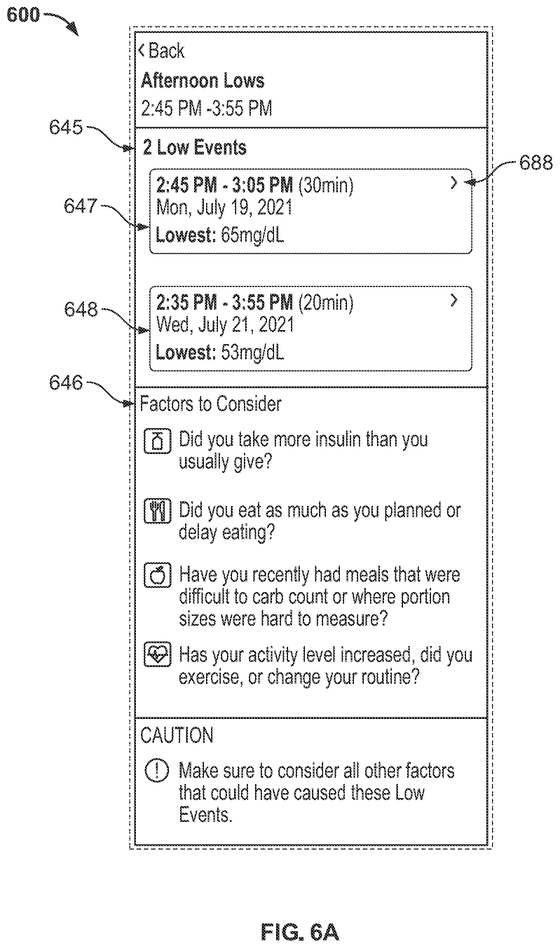

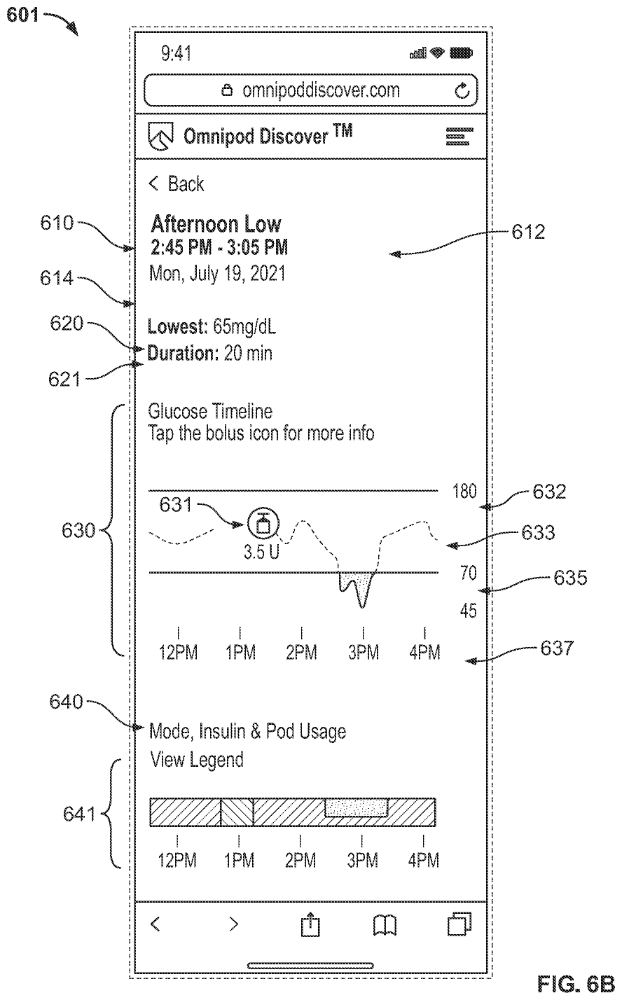

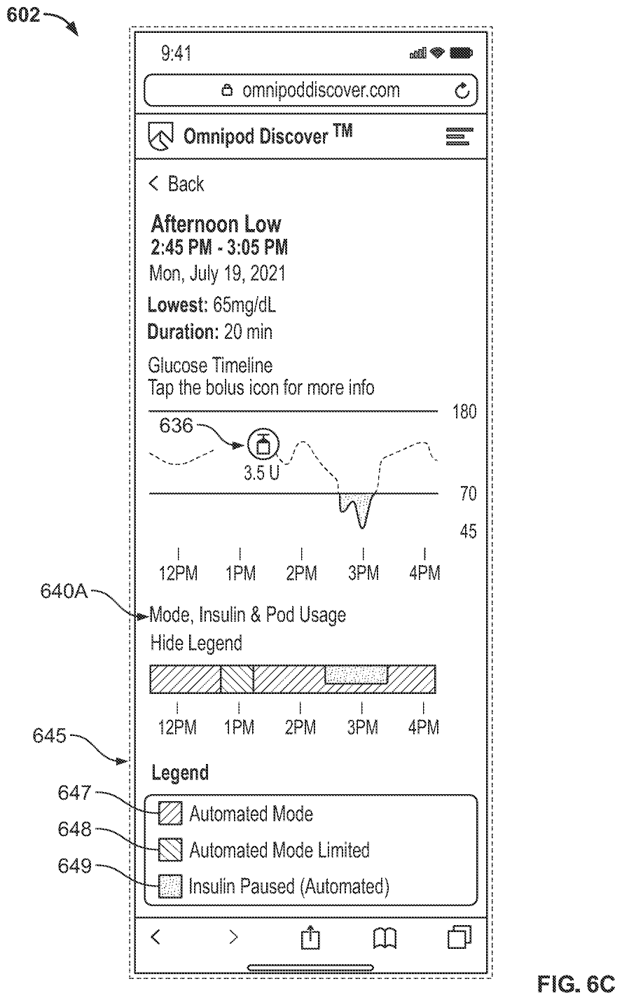

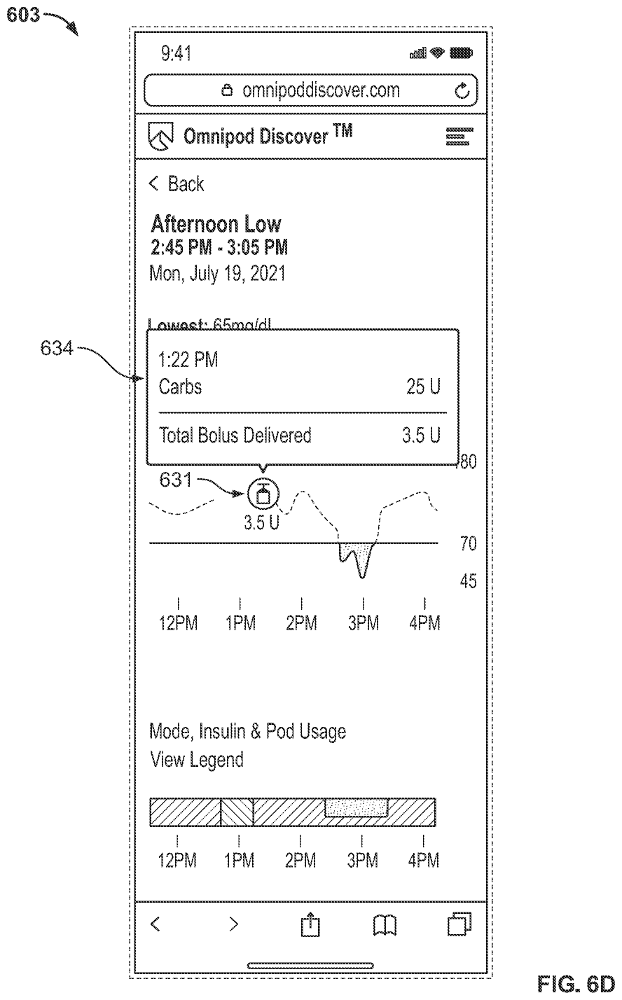

FIG. 6A illustrates an example of the graphical user interface that presents information related to multiple low event patterns that occurred in the afternoon with recommendations according to the subject matter described herein.

To access the information related to multiple high event patterns that occurred in the evening with recommendations, a user may select the “View Details” button 324 of FIG. 3 or 424 of FIG. 4 beneath the Afternoon low event patterns legend of the afternoon lows heading. In response to the selection, details of the multiple low event patterns are presented in the graphical user interface 600. The graphical user interface 600 presents the details of the 2 low event patterns under the low events header 645. While the number 2 is shown, the number shown may be greater than 2 if the number of low event patterns is greater and/or based on user settings (i.e., 2 is a minimum default but the user can set the number of low events to be presented to be greater than 2). Each of the low event patterns has their respective low event window 647 and 648. The low event window 647 shows the details of the Monday low event pattern and the low event window 648 shows the details of the Wednesday low event pattern. As shown, the details in each high event window 647 and 648 may include a lowest blood glucose measurement value obtained during the low event, a duration of the low event, such as 20 minutes, 30 minutes, 45 minutes, 1 hour or the like, as well as an operator, such as 688, that enables presentation of further details regarding the specific low event pattern. In addition, the graphical user interface 600 presents recommendations or “Factors to Consider” 646 based on the respective high event patterns. The recommendations 646 may be based on the lowest blood glucose measurement value obtained during the low event, the duration of the low event and/or other information.

FIG. 6B illustrates an example of a graphical user interface for presentation of a specific low blood glucose event according to the subject matter described herein.

In response to selection of user interface operator 688 of FIG. 6A or selection of a “view details” soft button such as one associated with Afternoon Lows, such as 324 of FIG. 3 or 424 of FIG. 4, the graphical user interface 601 of FIG. 6B may be presented to show details of the Afternoon Lows. In the example graphical user interface 601, the “Afternoon Low” heading 610 may be presented. Below the “Afternoon Low” heading 610 may be presented a time window 612 and a low event date 614 of the low event. The graphical user interface 601 may also include the lowest blood glucose measurement value that is measured during the time window 612 of the low event and the duration 621 of that lowest blood glucose measurement value.

Also presented in the graphical user interface 601 is a glucose timeline 630. The glucose timeline 630 is a graphic with a curve 633 showing a user's blood glucose measurement values over a timeline 637 which, in this example, is approximately 2:30 pm through to a short time after 7:00 pm, like 7:05 pm or so. The glucose timeline 630 also has borders that represent the high target blood glucose set point border 632 (in this example, 180 mg/dL) and the low target blood glucose set point border 635 (in this example, 70 mg/dL) and a bolus indicator 631. The curve 633 shows a segment color-coded red that extends below the low target blood glucose set point border 635. The red color-coding indicates that the user's blood glucose measurement values were in the hypoglycemic range that is below the user's low target blood glucose set point border 635. The bolus indicator 631 also includes a label showing the number of units of insulin that have been delivered. In the example, the bolus indicator 631 indicates that a bolus of 3.5 units (U) was delivered at approximately 1:15-1:30 pm. Also, the graphical user interface 601 includes a color-coded timeline mode indicator 61 that indicates modes of the drug delivery system during the timeline 637. The modes are color-coded but a legend may be presented by selection of the “View Legend” soft button 640.

FIG. 6C illustrates an example of the graphical user interface of FIG. 6B with a legend indicating drug delivery device mode and insulin usage of a drug delivery device according to the subject matter described herein. The graphical user interface 602 of FIG. 6C includes elements from the graphical user interface 601 of FIG. 6B that function in the same manner as described with reference to FIG. 6B so a detailed discussion of those elements is not repeated.

In response to the selection of the “View Legend” soft button 640, the graphical user interface 602 may present the legend 645 beneath soft button 640A that now presents “Hide Legend.” The legend 645 may present different modes that are color-coded such as an automated mode 647, an automated mode limited 648, and Insulin paused (automated) mode 649. In addition, or alternatively, the legend 645 may indicate when thresholds (both positive and negative thresholds) are reached or exceeded, such as in the case of “Insulin paused (automated)” 649.

The automated mode limited 648 color coded label that traverses approximately 12:45 pm-1:15 pm on the color-coded timeline mode indicator 641 of FIG. 6B. In an example, the data visualization algorithm described with reference to other examples may be operable to identify when blood glucose measurement values are missing during a time period, such as in gap 636. As mentioned above with reference to FIG. 1, blood glucose measurement values may be received from a continuous blood glucose monitor (CGM) at different time intervals during operation of the wearable drug delivery device. For example, some CGMs may output signals containing blood glucose measurement values every 5 minutes or even less, such as every minute. There are a number of different reasons why the data visualization application may not present a blood glucose measurement value at a particular time. For example, a CGM may fail to output a signal due to a faulty reading or loss of connectivity, the controller may not receive the signal from the CGM due to interference or is out of range, the signal is corrupted by interference or the like. Alternatively, the data visualization application (shown and described in another example) may receive a subsequent blood glucose measurement value that is so much higher or lower than the immediately preceding blood glucose measurement that it is considered an outlier and is not used. Whatever the reason for not receiving or using the blood glucose measurement value at the expected time, the data visualization algorithm is operable to indicate the missing blood glucose measurement value as a gap 636 in the curve 633 and also indicate in the color-coded timeline that the AID algorithm entered the automated mode limited mode.

FIG. 6D illustrates an example of the graphical user interface of FIG. 6B with a pop up window indicating bolus delivery time, bolus amount and estimated carbohydrates according to the subject matter described herein.

The graphical user interface 603 includes the bolus indicator 631 that has a label showing the number of units of insulin (i.e., 3.5 U) that have been delivered at approximately 1:15-1:30 μm. In response to selection of the bolus indicator 631, a pop-up window 634 may be presented that indicates a time (e.g., 1:22 pm) when the bolus was delivered, a number of carbohydrates provided to the bolus calculator of the AID algorithm (e.g., 25 U) and an amount of a total bolus dosage (e.g., 3.5 U) that was delivered to offset the carbohydrates. The pop-up window 634 may be presented for a predetermined period of time (e.g., 30 seconds, 1 minute, 3 minutes or the like). Alternatively, or in addition, the pop-up window 634 may be presented until the graphical user interface 603 is interacted with subsequent to the selection of the bolus indicator 631.

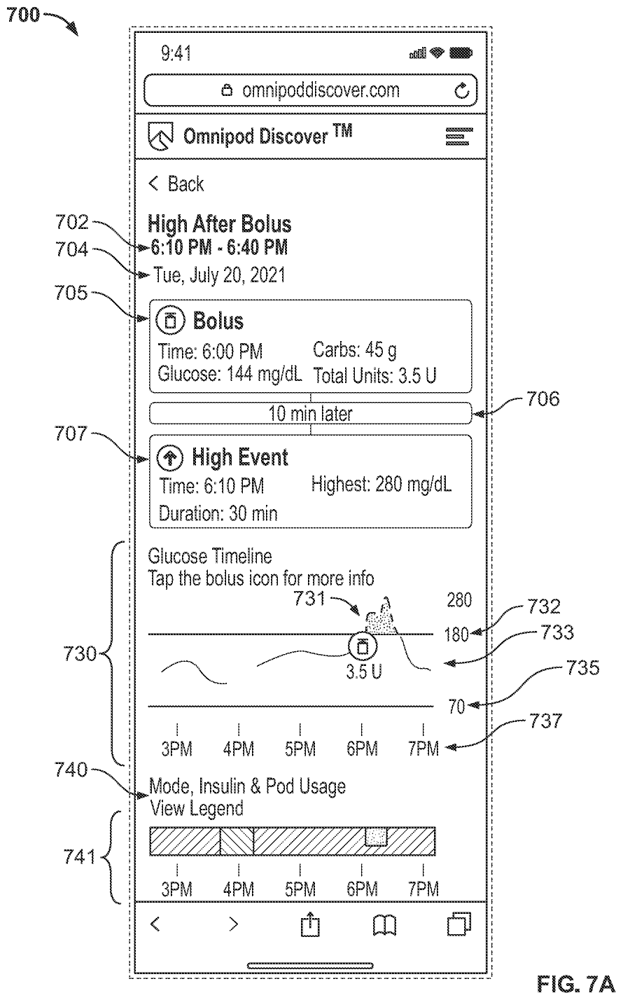

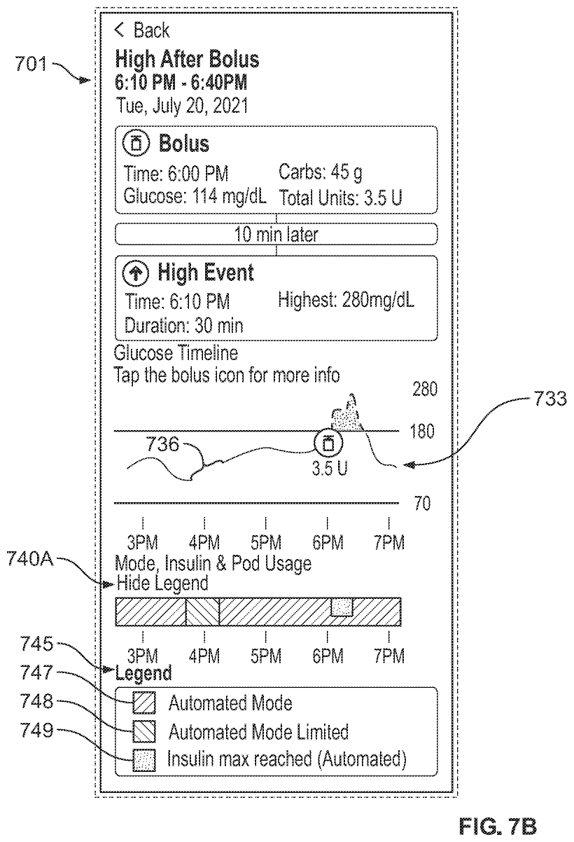

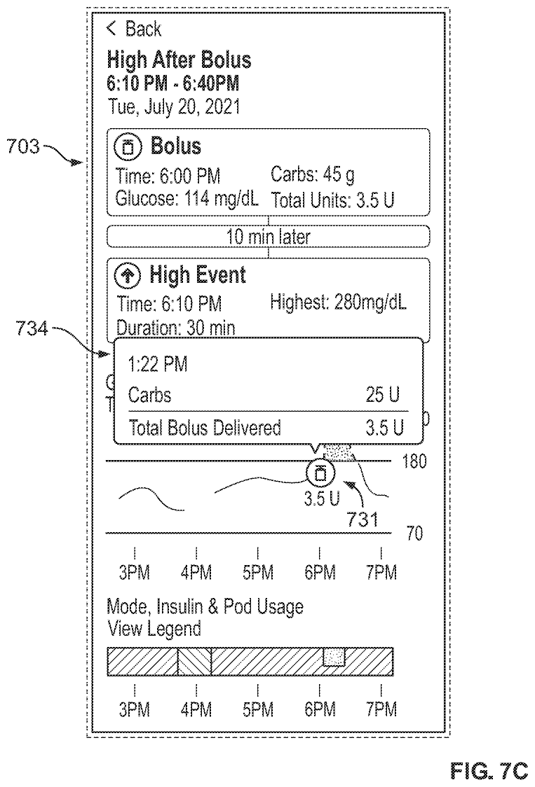

FIG. 7A illustrates an example of a graphical user interface for presentation of a high event after a bolus according to the subject matter described herein.

The graphical user interface 700 may present information related to a high event after the user administers a bolus, referred to as a “High After Bolus.” The graphical user interface 700 may present a high event after bolus duration 702, which in this example shows that the high event after bolus had a start time of 6:10 pm, an end time of 6:40 μm and a date 704 on which the high event after bolus occurred. The bolus details 705 includes information about the bolus, such as the time the bolus was delivered (e.g., 6:00 pm), a number of grams of carbohydrates (carbs) provided to the bolus calculator of the AID algorithm (e.g., 45 grams), a most recent blood glucose measurement value (e.g., “144 mg/dL”) and a total number of units that the bolus delivered (e.g., 3.5 U). A high event bolus delay indicator 706 shows an amount of time after a bolus, in this case, 10 minutes, that the high event after bolus occurred. The high event details indicator 707 shows details of the high event after bolus, such as the start time of the high event after bolus (i.e., 6:10 pm), a highest blood glucose measurement value during the high event (e.g., 280 mg/dL) and a duration of the high event after bolus (e.g., 30 minutes).

Also presented in the graphical user interface 700 is a glucose timeline 730. The glucose timeline 730 is a graphic with a curve 733 showing a user's blood glucose measurement values over a timeline 737 which, in this example, is approximately 2:30 pm through to a short amount of time after 7:00 pm, like 7:05 pm or so. The glucose timeline 730 also has borders that represent the high target blood glucose set point border 732 (in this example, 180 mg/dL) and the low target blood glucose set point border 735 (in this example, 70 mg/dL) and a bolus indicator 731. The bolus indicator 731 also includes a label showing the number of units of insulin that have been delivered. In the example, the bolus indicator 731 indicates that a bolus of 3.5 units (U) was delivered at approximately 6:00 pm. Also, the graphical user interface 700 includes a color-coded timeline mode indicator 741 that indicates modes of the drug delivery system during the timeline 737. The modes in the color-coded timeline mode indicator 741 are color-coded with a legend being presented below the indicator 741.

FIG. 7B illustrates an example of the graphical user interface of FIG. 7A with a legend indicating drug delivery device mode and insulin usage of a drug delivery device according to the subject matter described herein.

The graphical user interface 701 of FIG. 7B includes elements from the graphical user interface 700 of FIG. 7A that function in the same manner as described with reference to FIG. 7B so a detailed discussion of those elements is not repeated.

In response to the selection of the “View Legend” soft button 740, the graphical user interface 701 may present the legend 745 beneath soft button 740A that now presents “Hide Legend.” The legend 745 may present different modes that are color-coded such as an automated mode 747 and an automated mode limited 748. In addition, or alternatively, the legend 745 may indicate when thresholds (both positive and negative thresholds) are reached or exceeded, such as in the case of “Insulin max reached (automated)” 749.

The automated mode limited 748 color coded label that traverses approximately 3:45 pm-4:15 pm on the color-coded timeline mode indicator 741 of FIG. 7A. In an example, the data visualization algorithm described with reference to other examples may be operable to identify when blood glucose measurement values are missing during a time period, such as in gap 736. As mentioned above with reference to FIG. 1, blood glucose measurement values may be received from a continuous blood glucose monitor (CGM) at different time intervals during operation of the wearable drug delivery device. For example, some CGMs may output signals containing blood glucose measurement values every 5 minutes or even less, such as every minute. There are a number of different reasons why the data visualization application may not present a blood glucose measurement value at a particular time. For example, a CGM may fail to output a signal due to a faulty reading or loss of connectivity, the controller may not receive the signal from the CGM due to interference or is out of range, the signal is corrupted by interference or the like. Alternatively, the data visualization application (shown and described in another example) may receive a subsequent blood glucose measurement value that is so much higher or lower than the immediately preceding blood glucose measurement that it is considered an outlier and is not used. Whatever the reason for not receiving or using the blood glucose measurement value at the expected time, the data visualization algorithm is operable to indicate the missing blood glucose measurement value as a gap 736 in the curve 733 and also indicate in the color-coded timeline that the AID algorithm entered the automated mode limited mode.

FIG. 7C illustrates an example of the graphical user interface of FIG. 7A with a pop up window indicating bolus delivery time, bolus amount and estimated carbohydrates according to the subject matter described herein.

The graphical user interface 703 includes the bolus indicator 731 that has a label showing the number of units of insulin (i.e., 3.5 U) that have been delivered at approximately 4:30-4:40 pm. In response to selection of the bolus indicator 731, a pop-up window 534 may be presented that indicates a time when the bolus was delivered (e.g., 1:22 pm), a number of carbohydrates (e.g., 25 U) and an amount of a total bolus dosage (e.g., 3.5 U) that was delivered to offset the carbohydrates. The pop-up window 734 may be presented for a predetermined period of time (e.g., 30 seconds, 1 minute, 3 minutes or the like). Alternatively, or in addition, the pop-up window 534 may be presented until the graphical user interface 703 is interacted with subsequent to the selection of the bolus indicator 731.

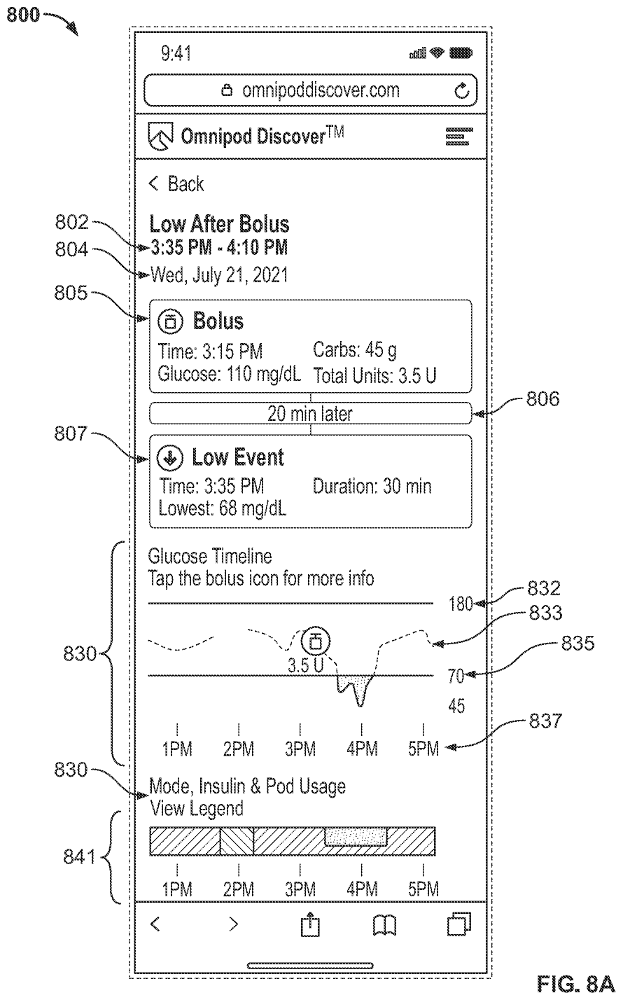

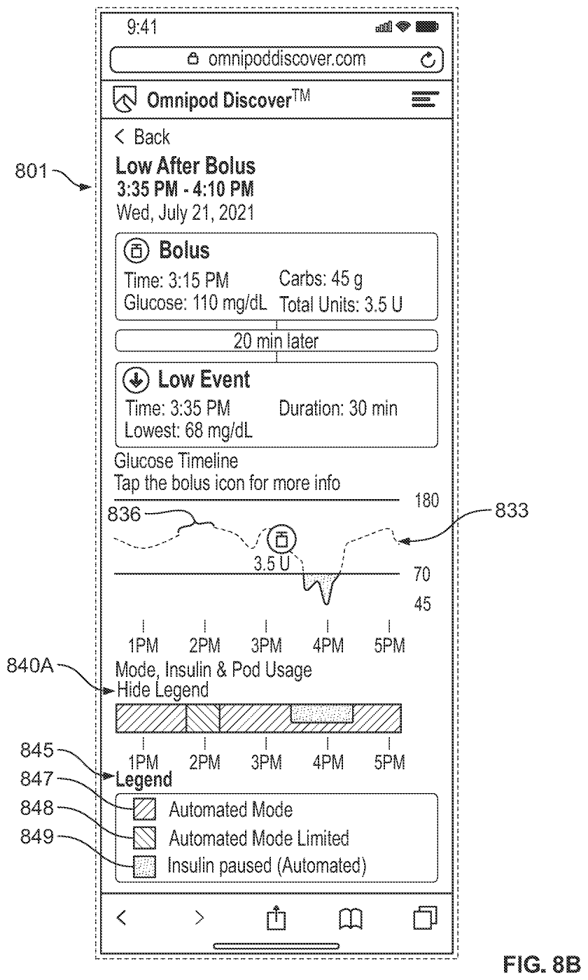

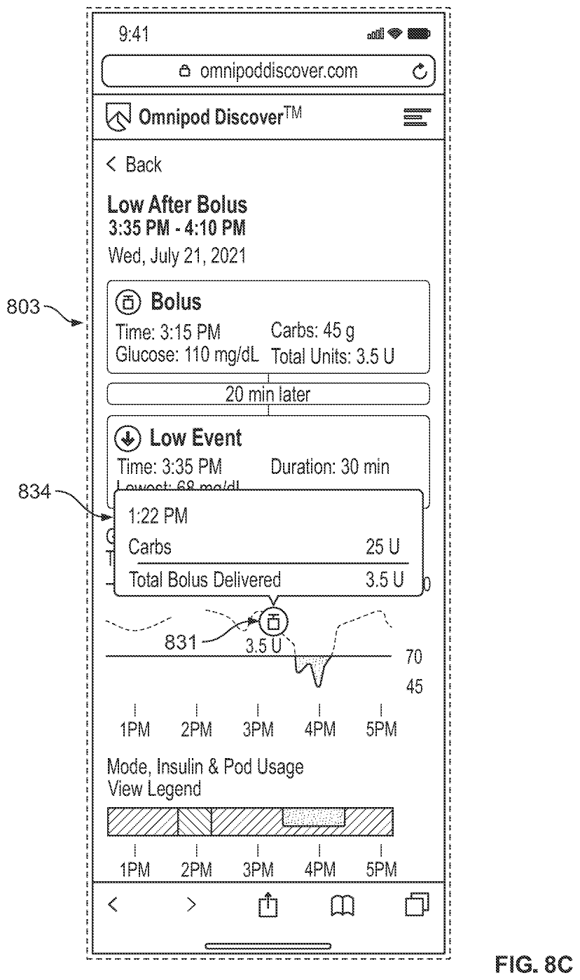

FIG. 8A illustrates an example of a graphical user interface for presentation of a low event after a bolus according to the subject matter described herein.

The graphical user interface 800 may present information related to a low event after the user administers a bolus, referred to as a “Low After Bolus.” The graphical user interface 800 may present a low event after bolus duration 802, which in this example shows that the low event after bolus had a start time of 3:35 pm, an end time of 4:10 μm and a date 804 on which the high event after bolus occurred. The bolus details 805 includes information about the bolus, such as the time the bolus was delivered (e.g., 3:15 pm), a number of grams of carbohydrates (carbs) provided to the bolus calculator of the AID algorithm (e.g., 45 grams), a most recent blood glucose measurement value (e.g., “110 mg/dL”) and a total number of units that the bolus delivered (e.g., 3.5 U). A low event bolus delay indicator 806 shows an amount of time after a bolus, in this case, 20 minutes, that the low event after bolus occurred. The low event details indicator 807 shows details of the high event after bolus, such as the start time of the high event after bolus (i.e., 3:35 pm), a highest blood glucose measurement value during the high event (e.g., 110 mg/dL) and a duration of the high event after bolus (e.g., 30 minutes).

Also presented in the graphical user interface 800 is a glucose timeline 830. The glucose timeline 830 is a graphic with a curve 833 showing a user's blood glucose measurement values over a timeline 837 which, in this example, is approximately 12:30 pm through to a short amount of time after 5:00 pm, like 5:05 pm or so. The glucose timeline 830 also has borders that represent the high target blood glucose set point border 832 (in this example, 180 mg/dL) and the low target blood glucose set point border 835 (in this example, 70 mg/dL) and a bolus indicator 831. The bolus indicator 831 also includes a label showing the number of units of insulin that have been delivered. In the example, the bolus indicator 831 indicates that a bolus of 3.5 units (U) was delivered at approximately 3:15 pm. Also, the graphical user interface 800 includes a color-coded timeline mode indicator 841 that indicates modes of the drug delivery system during the timeline 837. The modes in the color-coded timeline mode indicator 841 are color-coded with a legend being presented below the indicator 841.

FIG. 8B illustrates an example of the graphical user interface of FIG. 8A with a legend indicating drug delivery device mode and insulin usage of a drug delivery device according to the subject matter described herein.

The graphical user interface 801 of FIG. 8B includes elements from the graphical user interface 800 of FIG. 8A that function in the same manner as described with reference to FIG. 8B so a detailed discussion of those elements is not repeated.

In response to the selection of the “View Legend” soft button 840, the graphical user interface 801 may present the legend 845 beneath soft button 840A that now presents “Hide Legend.” The legend 845 may present different modes that are color-coded such as an automated mode 847 and an automated mode limited 848. In addition, or alternatively, the legend 845 may indicate when thresholds (both positive and negative thresholds) are reached or exceeded, such as in the case of “Insulin paused (automated)” 849.

The automated mode limited 848 color coded label that traverses approximately 1:45 pm-2:15 pm on the color-coded timeline mode indicator 841 of FIG. 7A. In an example, the data visualization algorithm described with reference to other examples may be operable to identify when blood glucose measurement values are missing during a time period, such as in gap 836. As discussed above with other examples that showed a gap in blood glucose measurement values, the data visualization algorithm is operable to indicate the missing blood glucose measurement value as a gap 836 in the curve 833 and also indicate in the color-coded timeline that the AID algorithm entered the automated mode limited mode.

FIG. 8C illustrates an example of the graphical user interface of FIG. 8A with a pop up window indicating bolus delivery time, bolus amount and estimated carbohydrates according to the subject matter described herein.

The graphical user interface 803 includes the bolus indicator 831 that has a label showing the number of units of insulin (i.e., 3.5 U) that have been delivered at 3:15 pm. In response to selection of the bolus indicator 831, a pop-up window 834 may be presented that indicates a time that the bolus was delivered (e.g., 1:22 pm), a number of carbohydrates (e.g., 25 U) and an amount of a total bolus dosage (e.g., 3.5 U) that was delivered to offset the carbohydrates. The pop-up window 834 may be presented for a predetermined period of time (e.g., 30 seconds, 1 minute, 3 minutes or the like). Alternatively, or in addition, the pop-up window 834 may be presented until the graphical user interface 803 is interacted with subsequent to the selection of the bolus indicator 831.

The graphical user interface 803 populated with at least 3 high event patterns and for at least 2 low event patterns, may be replaced with a weekly summary graphical user interface presented on a user device.

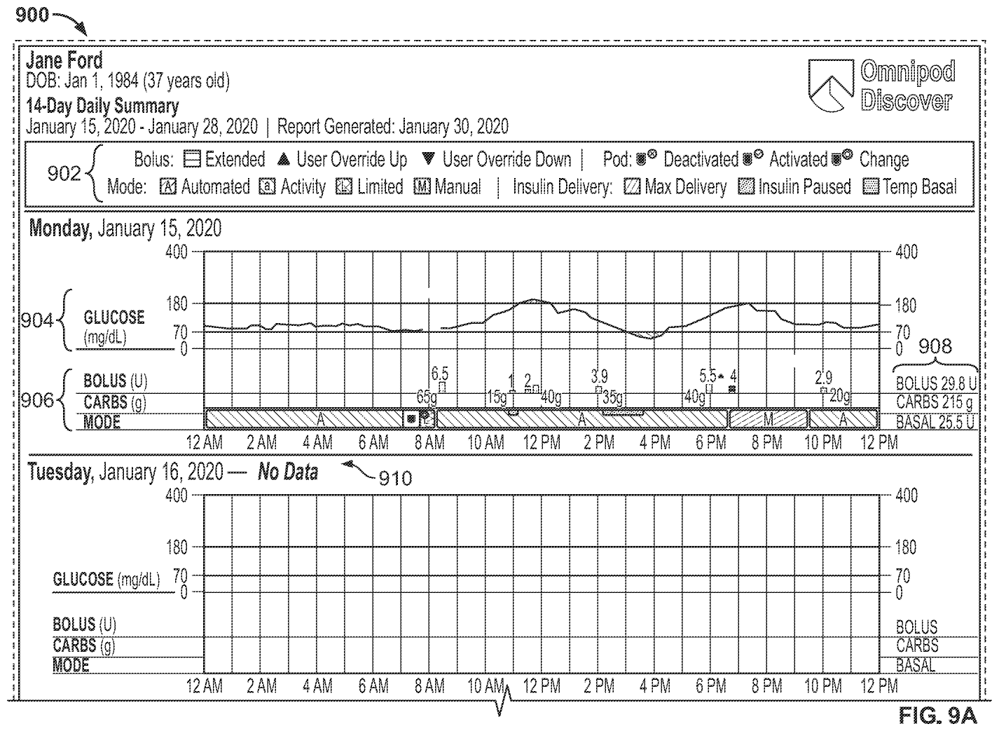

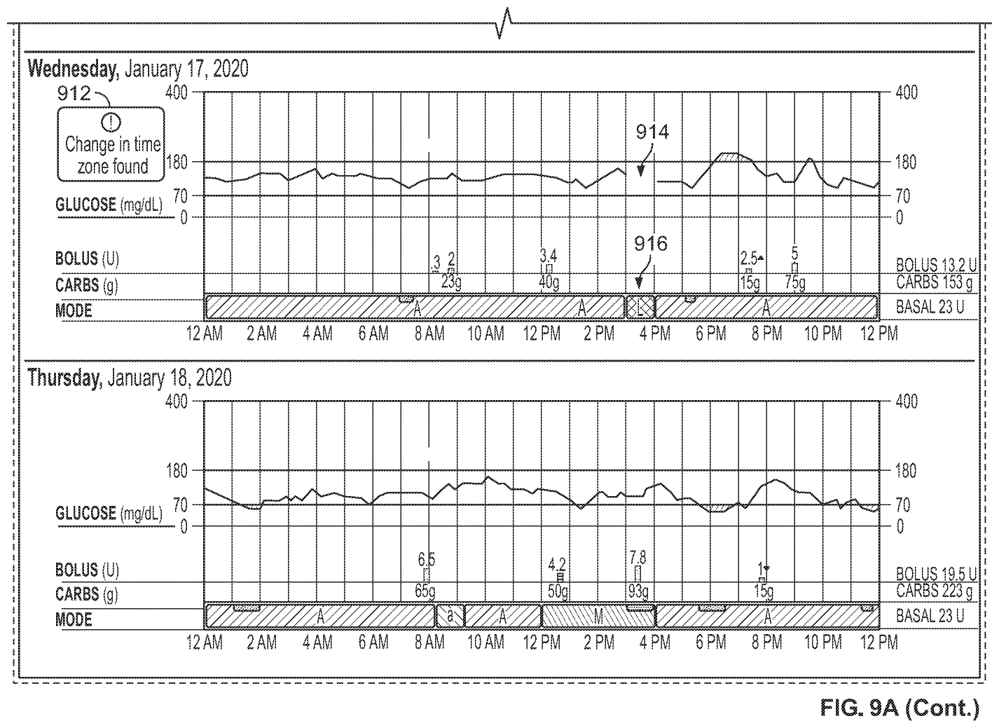

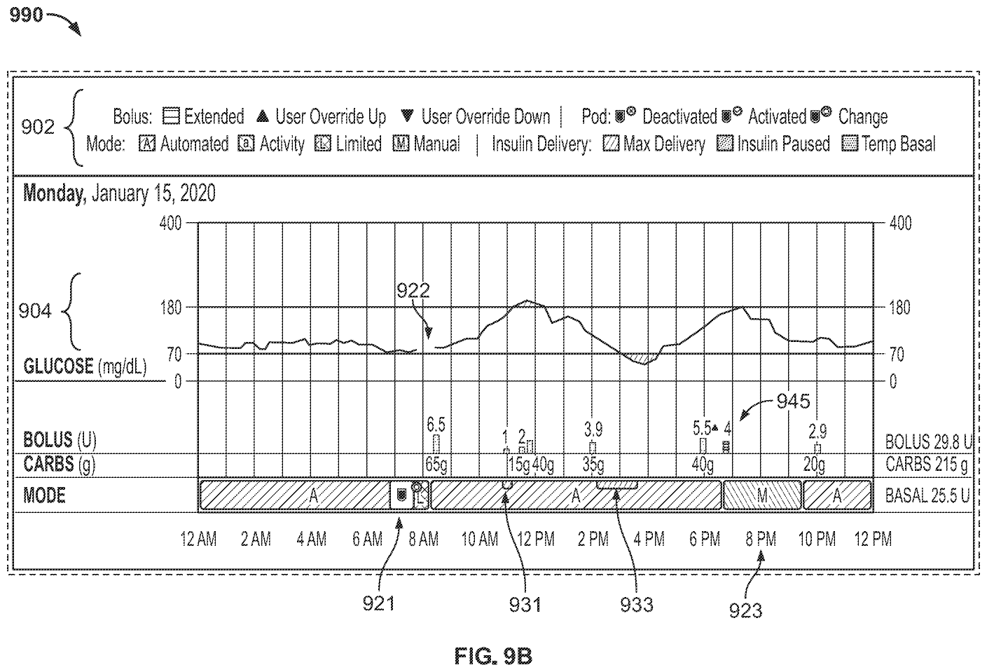

FIG. 9A illustrates an example of the graphical user interface providing a daily summary over a period of time according to the subject matter described herein.

The graphical user interface 900A may a comprehensive summary of a user's diabetes treatment program, carbohydrate intake and blood glucose measurements over a period of time. The legend 902 shows what the respective symbols illustrate for each of the respective day's information. The period of time may be 14 days and each day of the period of time is presented in a window that presents a timeline of a user's blood glucose measurement values 904, and a timeline 906 of a user's diabetes treatment program, carbohydrate intake and mode. The timeline 906 of a user's diabetes treatment program, carbohydrate intake and mode may have tiers of information that is aligned in time, such as bolus dosages, carbohydrates intake, and operational mode of a drug delivery device during basal delivery. The graphical user interface 900A may show totals 908 for the total amount of insulin delivered by bolus (i.e., 29.8 U), the carbohydrates ingested during the day (i.e., 215 g), and the amount of drug delivered via basal delivery (i.e., 25.5 U). The drug delivery device may have various operational modes and the controller may set limits for drug delivery based on a user's blood glucose measurement values, bolus delivered and carbohydrate information. The timeline 906 may present the respective operational mode of a drug delivery device during basal delivery and the set limits in a color-coded format for more intuitive interpretation according to data visualization principles.

Note that the above information is provided when the data is available. As shown for the Tuesday entry, a no data indicator 910 is presented. Instances where not data is available may be when the user's devices (i.e., controller, wearable drug delivery device or CGM) do not have connectivity to a network or the like.

In addition, as mentioned above, when there is a change in time zone indicated by time zone indicator 912, the data visualization may be operable to decline to calculate whether any high events or low events occurred and may indicate a gap 914 when the time zone change occurred. Therefore, the data visualization in the respective timeline 906 for the particular day may have limited information as indicated by 916 in the operational mode portion of the respective timeline.

FIG. 9B illustrates a detailed graphical user interface view example of a day example with a legend from the daily summary graphical user interface example of FIG. 9A according to the subject matter described herein.

The view 990 illustrates one day of the graphical user interface 900, which in this specific example is Monday. With reference to the legend 902, the different tiers (e.g., user's diabetes treatment program, carbohydrate intake and operational mode) of timeline 906 will be discussed in more detail. First in time is the indication 921 that the drug delivery device was changed. During the change, no blood glucose measurements were received or recorded as indicated by gap 922 in the glucose timeline 904. Next is the insulin delivery indication 931 that provides the user an indication that a maximum amount of insulin was delivered that also corresponded with a 1 U bolus at the same time. Subsequently, the insulin delivery indication 933 indicates that the AID algorithm paused delivery of insulin for a period of time after delivery of a bolus and consumption of a meal. The operational mode tier of timeline 906 also indicates that the controller implemented a manual mode (M) that corresponded with an extended bolus 945. The extended bolus dosage is indicated as 4 Units.

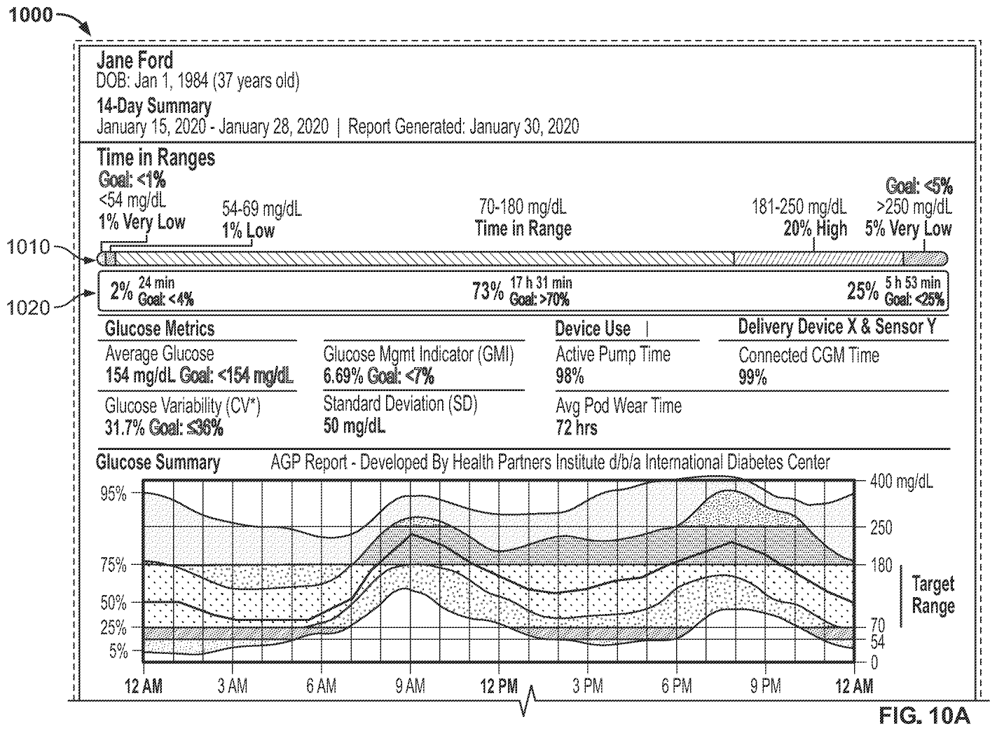

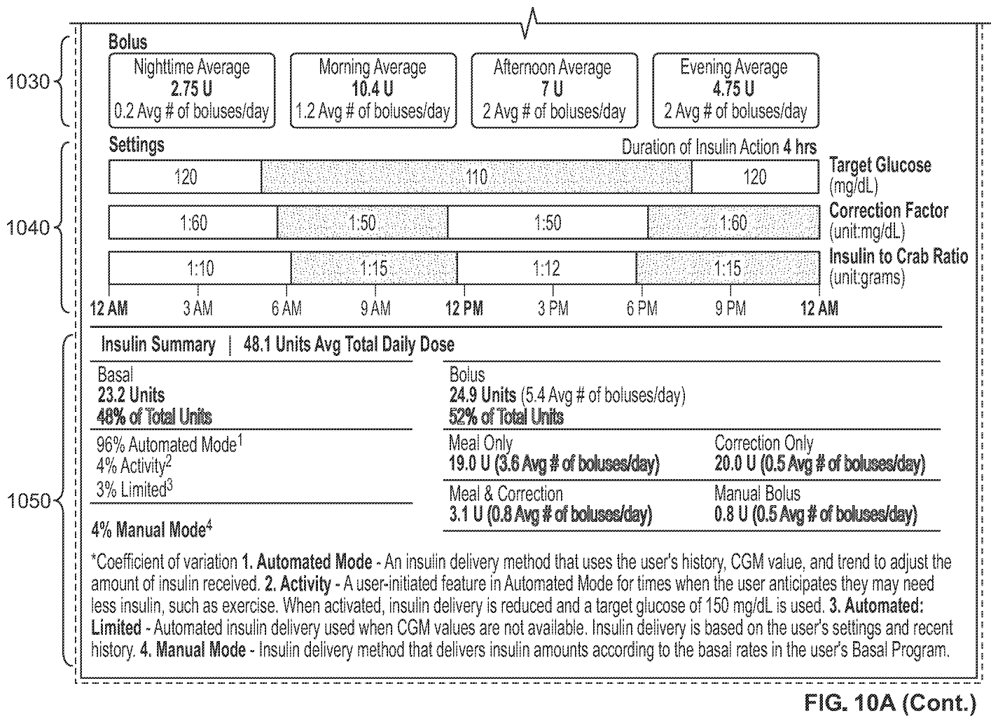

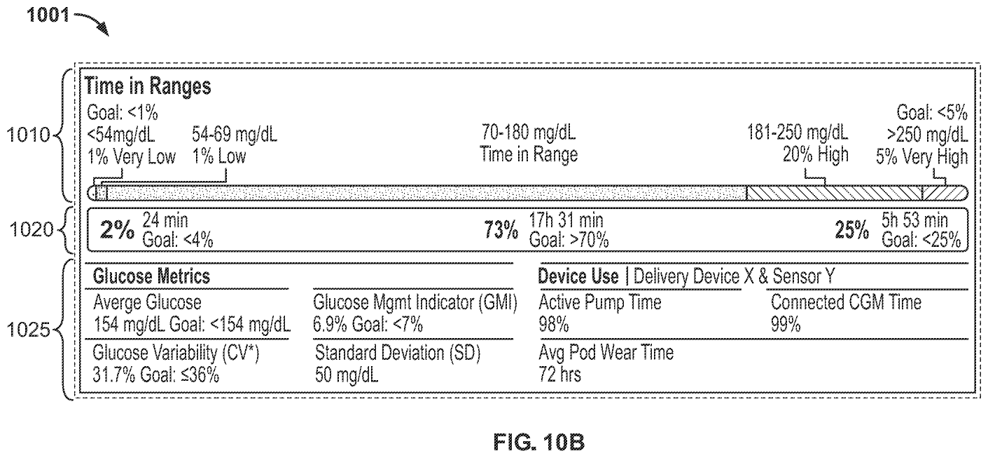

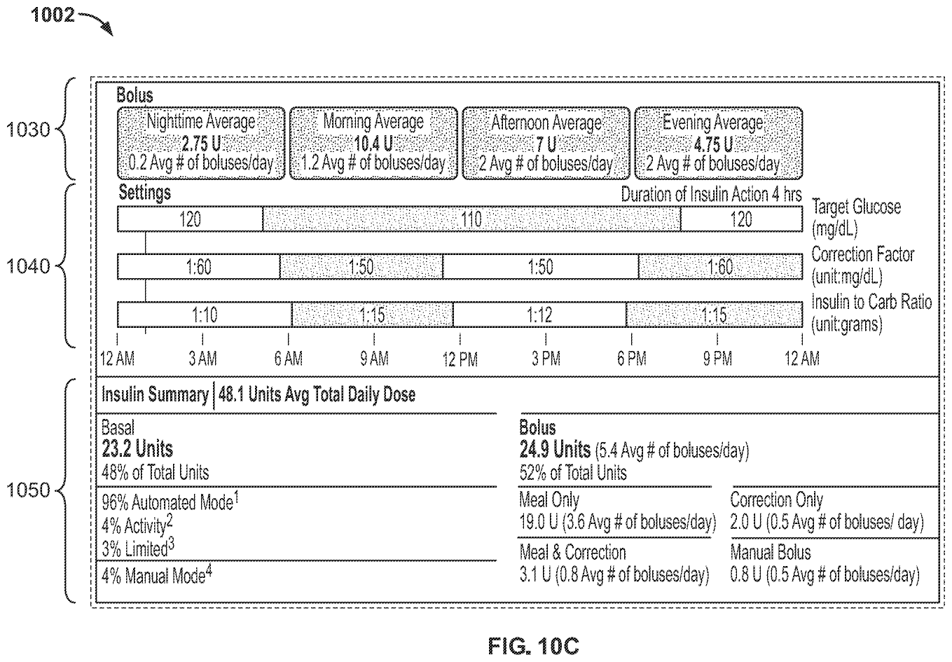

Other summary pages may also be presented. FIG. 10A illustrates an example of the graphical user interface providing a weekly summary based on time in range. The graphical user interface 1001 presents a horizontal timeline 1010 that provides a color coded indication of the percentage of time that the user remained in range of their low blood glucose target setpoint and their high blood glucose target setpoint, in this example 70-180 mg/dL. When the user is within range of their low blood glucose target setpoint and their high blood glucose target setpoint, this percentage of the summarized time (in this example, 14 days) is shown as green.

The bar 1020 provides a quick reference to indicate a low blood glucose range (i.e., 2%), in range (e.g., 73%) and high blood glucose range (e.g., 25%).

A bolus bar 1030 in the graphical user interface 1000 indicates an average bolus dosage during four time periods: nighttime (e.g., 2.75 U), morning (e.g., 10.4 U), afternoon (e.g., 7 U), and evening (e.g., 4.75 U) and additional information explained with reference to another figure.

Also presented in the graphical user interface 1000 is a settings bar 1040. The setting bars 1040 indicates average settings for a time of day in three setting categories: target glucose, correction factor and insulin to carb ratio. The setting bar 1040 indicates the times at which the respective settings for target glucose, correction factor and insulin to carb ratio are set. For example, the target glucose setting from 12 AM to approximately 5 AM is 120 mg/dL, from approximately 5 AM to approximately 8 PM is 110 mg/dL and from approximately 8 PM to 12 PM is 120 mg/dL. The correction factor like the insulin to carb ratio is a ratio and are represented in time segments in the same manner as the target glucose as shown in FIG. 10A.

The graphical user interface 1000 also presents an insulin summary 1050. The insulin provides delivered insulin information such as total daily dose (e.g., 48.1 U) that is further broken down into the number of units of insulin delivered by basal (e.g., 23.2 U) and the number of units of insulin delivered by bolus (e.g., 24.9 U). Detailed information related to the basal delivery is also presented such as the percentage of time the drug delivery device was controlled via automated mode, a percentage of time when the user anticipated activity and insulin delivery was decreased, or percentage of time when limited mode was engaged due to CGM data not being available. Bolus delivery also has detailed information presented such as a reason category that initiated the bolus delivery, such as a meal only, correction only, meal & correction and a manual mode delivery.

FIG. 10B illustrates a detailed graphical user interface view example of a weekly time in range from the weekly summary graphical user interface example of FIG. 10A according to the subject matter described herein.

In the view 1001 of graphical user interface 1000, the horizontal timeline 1010 also shows a percentage of the time when the user is low (e.g., in the range of blood glucose measurements equaling between 54-69 mg/dL) as a lighter-shade of red, and a further percentage of time when the user is very low (e.g., less than 54 mg/dL) by indicating a deep red. A user's highs are also presented as percentages and color coded on the horizontal timeline 1010. For example, when the user's blood glucose measurements are greater than the high blood glucose target setpoint, the horizontal timeline 1010 presents color coded percentage, in this example, 20% when the blood glucose measurements are between 181-25-mg/dL. The horizontal timeline 1010 also presents when the blood glucose measurement is very high (i.e., greater than 250%). The horizontal timeline 1010 indicates to the user the color coded percentage of the user's blood glucose measurements over the course of the summarized time period. For example, the longer a specific portion of the color coded horizontal timeline 1010 the greater the percentage of time that the user spent in that particular range of the blood glucose spectrum (e.g., 54 mg/dL to 250 mg/dL).

In addition to the bar 1020 providing a quick reference to indicate a low blood glucose range (i.e., 2%), in range (e.g., 73%) and high blood glucose range (e.g., 25%), the bar 1020 also provides a percentage goal for each of the ranges as well as an amount of time in each range to provide an indication of a user's conformance to their diabetes treatment plan. The percentage goal for each of the respective ranges is tunable based on a user's diabetes treatment plan.

The view 1001 also provides additional information 1025 under a “Glucose Metrics” heading, the information 1025 related to the user's blood glucose measurement values for the summarized time period, such as average glucose measurement value (and goal), glucose management indicator (GMI) (and goal), glucose variability (CV*)(and goal) and standard deviation, may be presented.

The additional information 1025 in view 1001 may further provide under a “Device Use” heading, information related to the user's drug delivery device and CGM. For example, the specific device model name may be listed, such as delivery device X (for a drug delivery device) and sensor Y (for a CGM). Statistics for the summarized time period, such as active pump time, connected CGM time, and average drug delivery device wear time, may also be presented.

FIG. 10C illustrates a detailed graphical user interface view example of a weekly bolus and insulin summary from the weekly summary graphical user interface example of FIG. 10A according to the subject matter described herein.

Also presented is an amount of insulin delivered per the number of boluses per day for each of a nighttime (e.g., 0.2 Avg # of boluses/day) a morning (e.g., 1.2 Avg # of boluses/day), an afternoon (e.g., 2 Avg # of boluses/day), and an evening (e.g., 2 Avg # of boluses/day).

The setting bar 1040 indicates the times at which the respective settings for target glucose, correction factor and insulin to carb ratio are set. For example, the target glucose setting from 12 AM to approximately 5 AM is 120 mg/dL, from approximately 5 AM to approximately 8 PM is 110 mg/dL and from approximately 8 PM to 12 PM is 120 mg/dL. The correction factor like the insulin to carb ratio is a ratio and are represented in time segments in the same manner as the target glucose as shown in FIG. 10A.

The insulin summary 1050 provides detailed insulin information related to the delivery of insulin to the user. The presentation of the insulin summary 1050 is a tree hierarchy of insulin delivery of a statistical day from total average insulin delivered daily and the breakdown to basal/bolus split. A part of the insulin summary 1050, for example, includes information regarding the basal delivery (i.e., a basal breakdown). Basal delivery information is presented as a percentage of time the drug delivery device was controlled via automated mode, a percentage of time when the user anticipated activity and insulin delivery was decreased, or percentage of time when limited mode was engaged due to CGM data not being available. Within the basal breakdown of average basal units delivered and the supported percent of pump modes (e.g., automated, Hypoprotect (as a feature), limited and manual delivery) may also be presented.

The insulin summary 1050 also includes detailed information related to bolus delivery (e.g., a bolus break down) is also presented and includes, for example, a reason category indicating what event initiated the bolus delivery, such as deliver of a bolus to address consumption of a meal only, a correction bolus to get the user's blood glucose back in range, a combination of meal & correction and a manual mode delivery. Within the bolus breakdown, the average bolus delivered, with additional breakdowns to types of boluses (e.g., meal, override, meal & correction & manual).

While insulin is referred to often in the above discussion as the therapeutic drug or liquid drug delivered by the wearable drug delivery device, insulin may be replaced by other drugs analogous to insulin, by combinations of drugs, or by formulations which may include, for example, glucagon or glucagon-like peptides, or the like, as well as other drugs for other purposes, such as chemotherapy drugs, pain relief drugs, such as opioids and narcotics, or the like, fertility drugs and other drugs that require cautious monitoring and delivery.

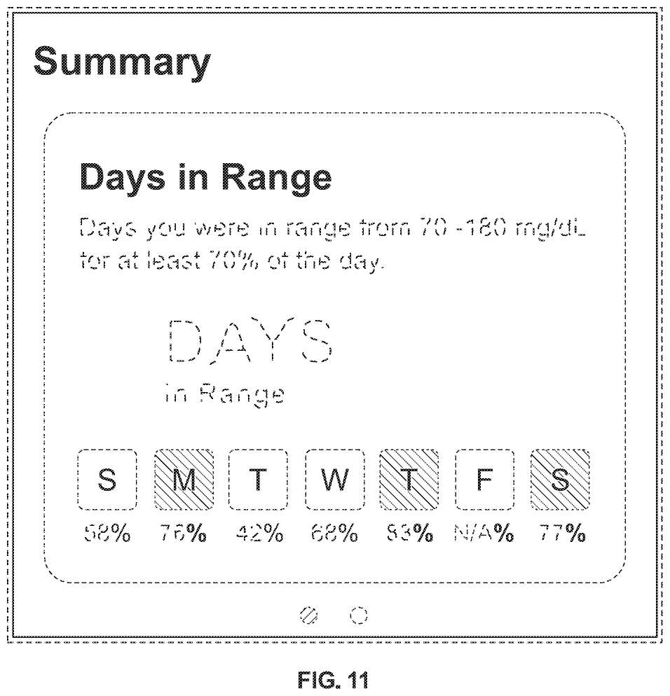

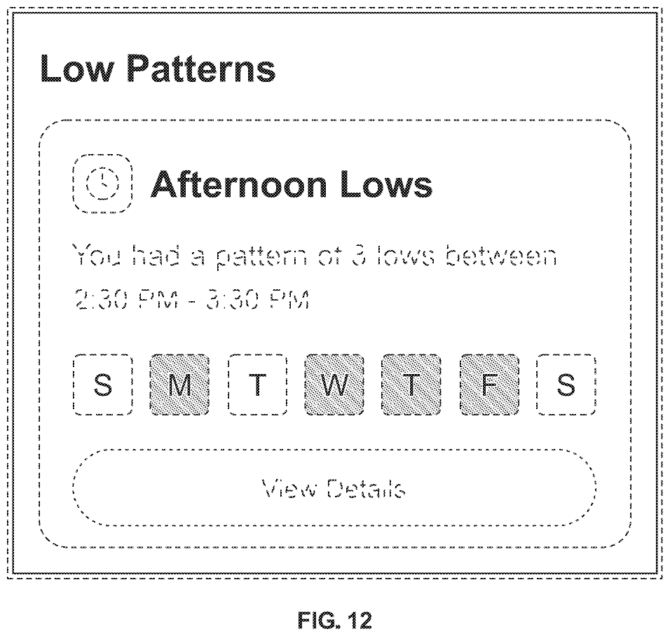

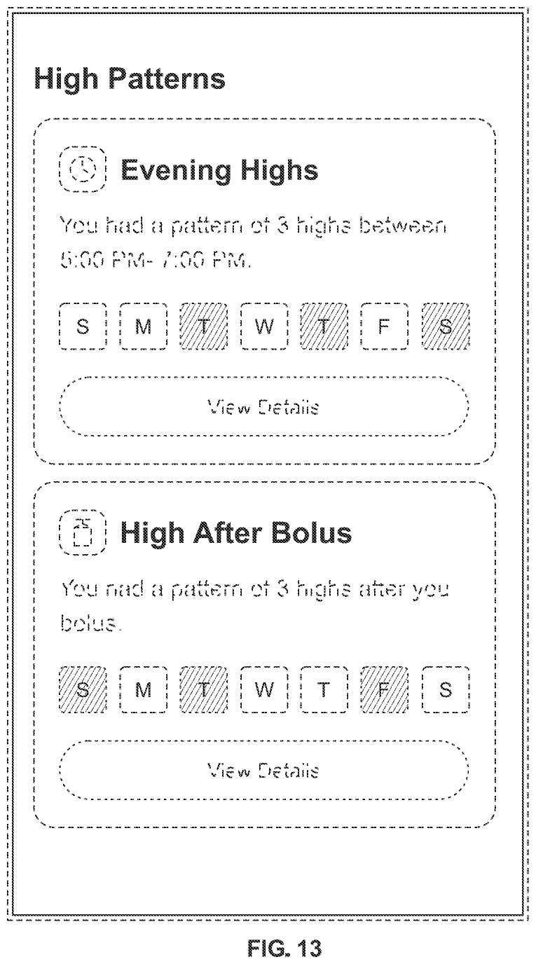

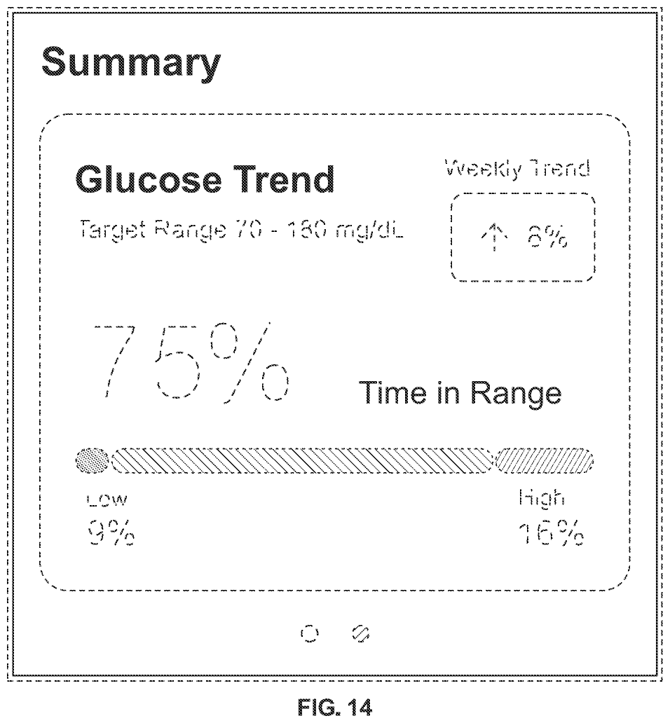

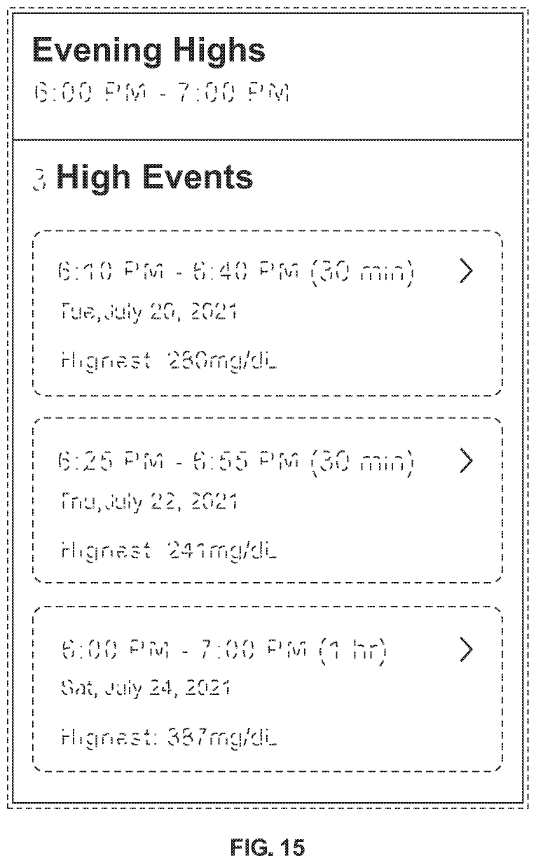

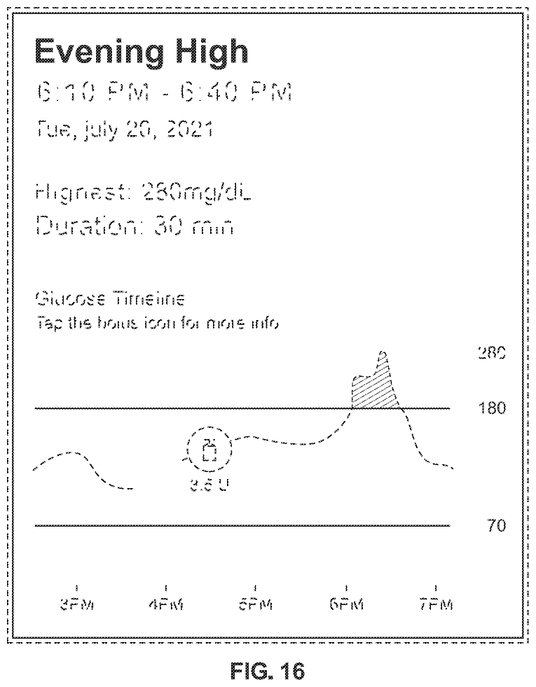

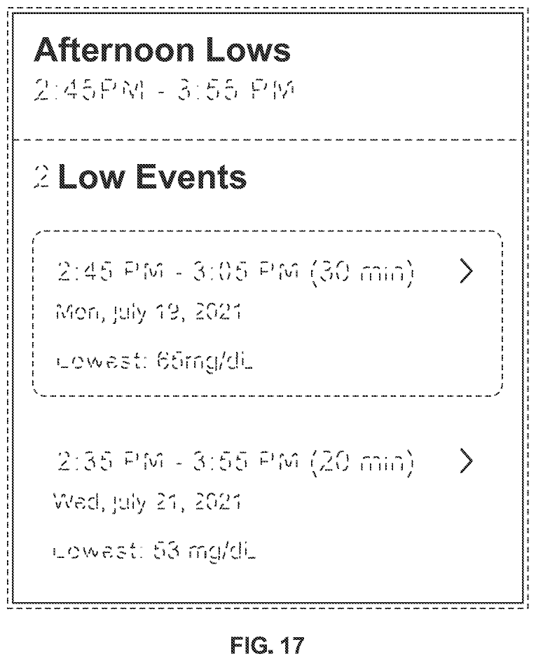

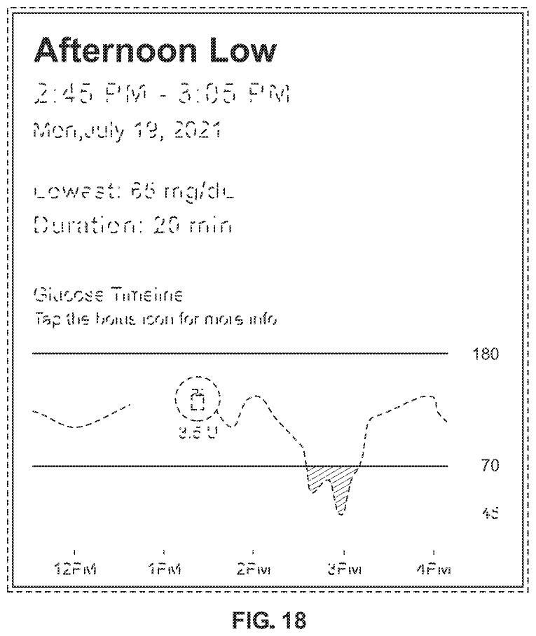

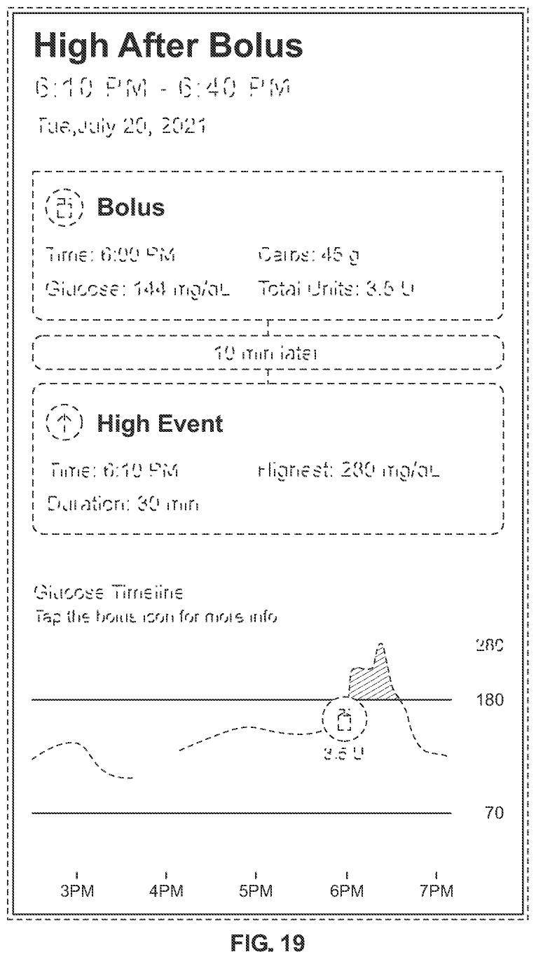

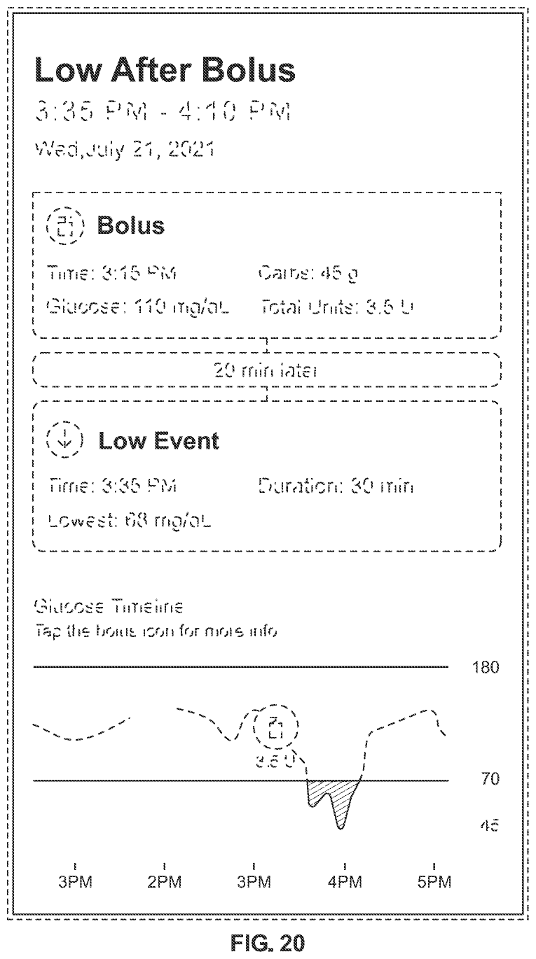

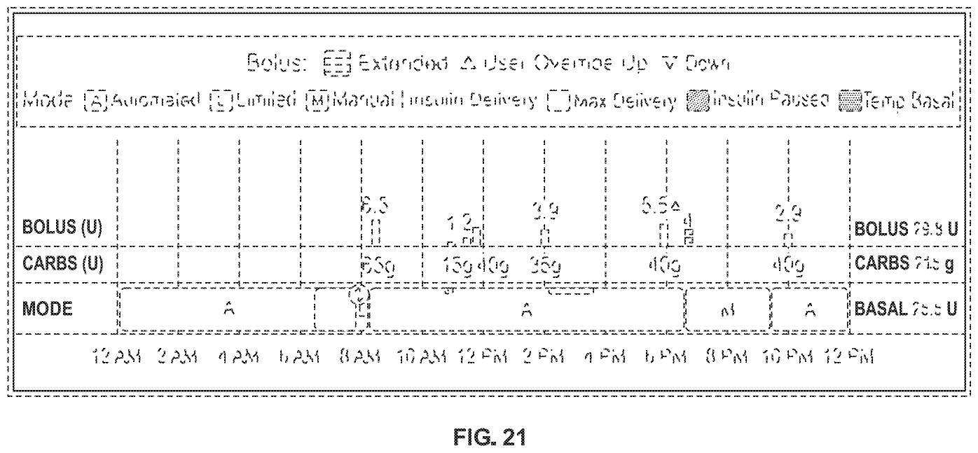

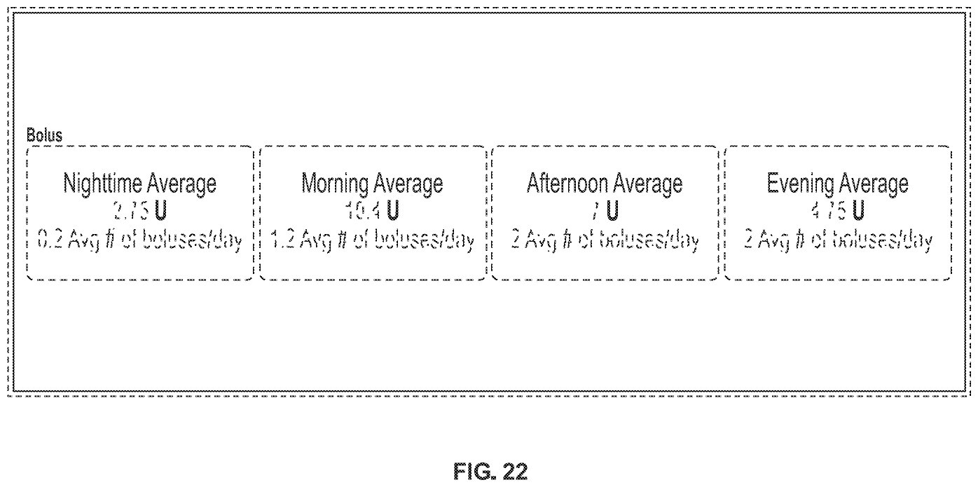

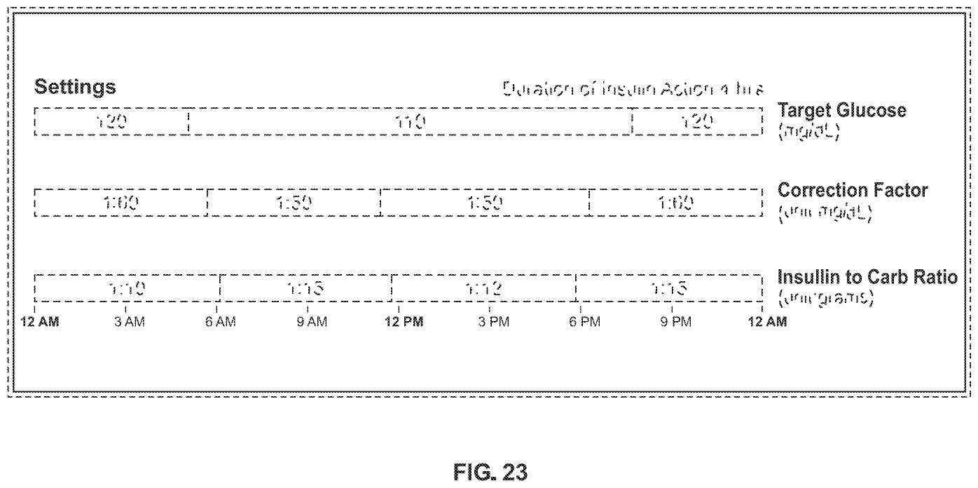

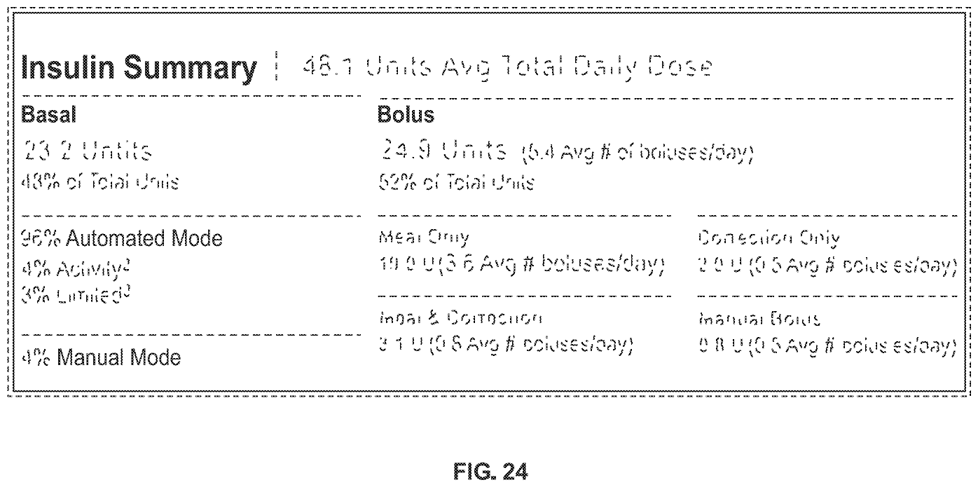

FIGS. 11-24 provide views of ornamental features of a graphical user interface. For example, the example graphical user interface depicted in FIG. 11 may present a summary of days in range. FIG. 12 illustrates a graphical user interface that may present headings for “low patterns” and “afternoon lows.” FIG. 13 illustrates a graphical user interface that may present headings for a “high patterns,” “evening highs” and “high after bolus.” FIG. 14 illustrates a graphical user interface that may present headings for a summary of glucose trend of a user. FIG. 15 illustrates a graphical user interface that may present headings for “evening highs” and “3 high events.” FIG. 16 illustrates a graphical user interface that may present an “evening high” heading. FIG. 17 illustrates a graphical user interface that may present headings for “afternoon lows” and “2 low events.” FIG. 18 illustrates a graphical user interface that may present an “afternoon low” heading. FIG. 19 illustrates a graphical user interface that may present a heading of a “high after bolus,” and subheadings of a “bolus” and a “high event.” FIG. 20 illustrates a graphical user interface that may present a heading of a “low after bolus,” and subheadings of a “bolus” and a “low event.” FIG. 21 illustrates a graphical user interface that may present a summary of delivery of a bolus, carbohydrates (CARBS) and a mode of the drug delivery device as well as respective units of the bolus(es) and carbohydrates. FIG. 22 illustrates a graphical user interface that may present a summary of a bolus arranged as nighttime average with units, a morning average with units, an afternoon average with units and an evening average with units. FIG. 23 illustrates a graphical user interface that may present a summary of settings arranged as a 24-hour timeline with target glucose, correction factor and insulin to carb ratio. FIG. 24 illustrates a graphical user interface that may present an insulin summary of basal and bolus with presentation of an automated mode subheading and manual mode subheading under a basal heading. A boundary of the graphical user interface is shown in solid lines with ornamental features shown in solid lines with other features shown in dashed lines, but that may also have been introduced as solid lines.

The various elements of the devices, apparatuses or systems as previously described with reference to the FIGs. may include various hardware elements, software elements, or a combination of both. Examples of hardware elements may include structural members, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

What is claimed is:

1. A non-transitory computer readable medium embodied with programming instructions that when executed cause a processor to:
access blood glucose measurement values and insulin data from a data warehouse memory;
identify blood glucose measurement values that exceed an upper target blood glucose set point as a high events and blood glucose measurement values that are below a lower target blood glucose set point as low events;
apply a high event overlap rule set to the high events and a low event overlap rule set to the low events;
based on the application of the high event overlap rule set to the high events and the low event overlap rule to the low events, identify overlapping high events and overlapping low events for analysis;
identify as a high event pattern of the overlapping high events those high events that fall within a set high event overlap duration and day range;
identify as a low event pattern of the overlapping low events those low events that fall within a set low event overlap duration and day range;
apply a respective pattern weight to each identified high event and each identified low event based on criteria specific to high events and specific to low events; and
populate a graphical user interface with a number of high event patterns and for another number of low event patterns.

2. The non-transitory computer readable medium of claim 1, further embodied with programming instructions that when executed cause the processor, when identifying blood glucose measurement values that exceed the upper target blood glucose set point as high events, to:
obtain a user's upper target blood glucose set point from user settings stored in memory.

3. The non-transitory computer readable medium of claim 1, further embodied with programming instructions that when executed cause the processor, when identifying blood glucose measurement values that are below the lower target blood glucose set point as low events, to:
obtain a user's lower target blood glucose set point from user settings stored in memory.

4. The non-transitory computer readable medium of claim 1, further embodied with programming instructions that when executed cause the processor, when generating corresponding high event patterns, to:
apply high event overlap rules that include evaluating high events that overlap according to a high event duration rule and extend across three days.

5. The non-transitory computer readable medium of claim 1, further embodied with programming instructions that when executed cause the processor, when generating corresponding low event patterns, to:
apply low event overlap rules that include evaluating high events that overlap according to a low event duration rule and extend across three days.

6. The non-transitory computer readable medium of claim 1, further embodied with programming instructions that when executed cause the processor to:
obtain a weekly blood glucose measurement values and insulin data from the data warehouse memory.

7. The non-transitory computer readable medium of claim 6, further embodied with programming instructions that when executed cause the processor to:
replace the graphical user interface populated with at least 3 high event patterns and for at least 2 low event patterns with a weekly summary graphical user interface based on the weekly blood glucose measurement values and the insulin data from the data warehouse memory.

8. A non-transitory computer readable medium embodied with programming instructions that when executed cause a processor to:
access blood glucose measurement values and insulin data from a memory;
identify blood glucose measurement values that exceed an upper target blood glucose set point as high events and blood glucose measurement values that are below a lower target blood glucose set point as low events;
identify high event patterns in the high events that overlap and low event patterns in the low events that overlap, where in the overlap is determined based on a window of time;
apply a bolus event rule set to the identified overlapping high event patterns and identified overlapping low event patterns;
based on the application of the bolus event rule set to the respective identified overlapping low events and the respective identified low events, identify bolus events that correspond to respective identified high events and respective identified low events for analysis;
append the identified bolus events to a corresponding identified high event or a corresponding identified low event in the memory; and
populate a graphical user interface with a number of low event patterns with appended bolus events and with another, different number of high events with appended bolus events.

9. The non-transitory computer readable medium of claim 1, further embodied with programming instructions that when executed cause a processor, when identifying high event patterns in the high events that overlap, where in the overlap is determined based on a window of time; is operable to:
sum the blood glucose measurement values during a respective high event duration and dividing the sum by the total number of identified high events during the window of time encompassing the respective high event duration.

10. The non-transitory computer readable medium of claim 1, further embodied with programming instructions that when executed cause a processor, when identifying low event patterns in the low events that overlap, where in the overlap is determined based on a window of time; is operable to:
sum the blood glucose measurement values during the respective low event duration and dividing the sum by the total number of identified low events during the window of time encompassing the respective low event duration.

11. A system, comprising:
a memory operable to store a user's continuous blood glucose monitor data and data from a user's personal diabetes management device, wherein the user's continuous blood glucose monitor data includes blood glucose measurement values and the data from the user's personal diabetes management device includes insulin delivery data;
a data analysis processor, including circuitry operable to execute programming code stored in the memory, is operable to:
obtain blood glucose measurement values and insulin delivery data and the data from the user's personal diabetes management device for a predetermined time period;
identify in the normalized data blood glucose measurement values that exceed an upper target blood glucose set point as high events and blood glucose measurement values that are less than a lower target blood glucose set point as low events;
apply a high event overlap rule set to the high events and a low event overlap rule set to the low events;
based on the application of the high event overlap rule set to the high events and the low event overlap rule to the low events, identify overlapping high events and overlapping low events for analysis;
identify as a high event pattern of the overlapping high events those high events that fall within a set high event overlap duration and day range;
identify as a low event pattern of the overlapping low events those low events that fall within a set low event overlap duration and day range;
apply a respective pattern weight to each identified high event and each identified low event based on criteria specific to high events and specific to low events; and
populate a graphical user interface with a number of high event patterns and with another, different number of low event patterns.

12. The system of claim 11, wherein the data analysis processor is further operable, when identifying blood glucose measurement values that are greater than the lower target blood glucose set point as low events, to:
obtain a user's upper target blood glucose set point from user settings stored in memory.

13. The system of claim 11, wherein the data analysis processor is further operable, when identifying blood glucose measurement values that are less than the lower target blood glucose set point as low events, to:
obtain a user's lower target blood glucose set point from user settings stored in memory.

14. The system of claim 11, wherein the data analysis processor is further operable, when generating corresponding high event patterns, to:
apply high event overlap rules that include evaluating high events that overlap according to a high event duration rule and extend across three days.

15. The system of claim 11, wherein the data analysis processor is further operable, when generating corresponding low event patterns, to:
apply low event overlap rules that include evaluating high events that overlap according to a low event duration rule and extend across three days.

16. The system of claim 11, wherein the data analysis processor is further operable to:
obtain a weekly blood glucose measurement values and insulin data from the data warehouse memory.

17. The system of claim 16, wherein the data analysis processor is further operable to:
replace the graphical user interface populated with at least 3 high event patterns and for at least 2 low event patterns with a weekly summary graphical user interface.

18. The system of claim 11, wherein the data extraction, transformation and loading circuitry when normalizing the user's continuous blood glucose monitor data and data from the user's personal diabetes management device is operable to:
access the data received from the user's continuous blood glucose monitor and the user's person diabetes management device,
extract blood glucose measurement data from the user's continuous blood glucose monitor data and the data from the user's personal diabetes management device; and
format the data for identification of the high event patterns and low event patterns.
